(19) United States
(12) Patent Application Publication
(10) Pub. No.: US 2018/0311473 A1
(43) Pub. Date: Nov. 1, 2018

(54) HYBRID FLUID/MECHANICAL ACTUATION AND TRANSSEPTAL SYSTEMS FOR CATHETERS AND OTHER USES

(71) Applicant: Project Moray, Inc., Belmont, CA (US)

(72) Inventors: Keith Phillip Laby, Oakland, CA (US); Miles D. Alexander, Fremont, CA (US); Mark D. Barrish, Belmont, CA (US)

(21) Appl. No.: 15/963,004

(22) Filed: Apr. 25, 2018

Related U.S. Application Data
(60) Provisional application No. 62/489,826, filed on Apr. 25, 2017.

(51) Int. Cl. A61M 25/01; A61M 25/10; A61B 18/14; A61F 2/24

(52) U.S. Cl. CPC A61M 25/0147; A61M 25/10181; A61B 2017/00575; A61F 2/2427; A61B 18/1492

(57) ABSTRACT

Medical devices, systems, and methods for catheter-based structural heart therapies, including positioning of prosthetic mitral valves, make use of catheter structures that can flex when advanced over a pre-bent guidewire. Telescoping transseptal access systems use steering segments that are disposed proximal of a relatively rigid catheter segment (the segment optionally supporting a prosthetic valve) by engaging tissue adjacent the right atrium near the proximal end of the valve, and by telescoping a relatively rigid needle guide distally from the valve across the right atrium to engage tissue of the fossa ovalis. Hybrid pull-wire/balloon articulation systems may optionally employ relatively stiff pull-wire articulation within the right atrium, and relatively flexible balloon articulation systems within the left atrium. More generally, hybrid systems may have catheter systems with pullwires or movable sheath, along with fluid drive and robotic control components.

### CROSS REFERENCE TO RELATED APPLICATIONS

The present application claims the benefit of U.S. Provisional Application Ser. No. 62/489,826, filed on Apr. 25, 2017, which is incorporated by reference herein in its entirety for all purposes.

### FIELD OF THE INVENTION

In general, the present invention provides improved medical devices, systems, and methods. In exemplary embodiments, the invention provides improved structures and methods for traversing the septal wall, with the technologies being particularly well suited for accessing target tissues of the heart for treatment and/or diagnosis using a fluid-driven articulation balloon array that can help shape, steer and/or advance a catheter, guidewire, or other elongate flexible structure.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging. Alternative minimally invasive surgical technologies include robotic surgery, and robotic systems for manipulation of flexible catheter bodies from outside the patient have also previously been proposed. Some of those prior robotic catheter systems have met with challenges, in-part because of the difficulties in accurately controlling catheters using pull-wires. While the potential improvements to surgical accuracy make these efforts alluring, the capital equipment costs and overall burden to the healthcare system of these large, specialized systems is a concern.

A new technology for controlling the shape of catheters has recently been proposed which may present significant advantages over pull-wires and other known catheter articulation systems. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), an articulation balloon array can include subsets of balloons that can be inflated to selectively bend, elongate, or stiffen segments of a catheter. These articulation systems can use pressure from a simple fluid source (such as a pre-pressurized canister) that remains outside a patient to change the shape of a distal portion of a catheter inside the patient via a series of channels in a simple multi-lumen extrusion, providing catheter control beyond what was previously available often without having to resort to a complex robotic gantry, without pull-wires, and even without motors. Hence, these new fluid-driven catheter systems appear to provide significant advantages.

Despite the advantages of the newly proposed fluid-driven catheter system, as with all successes, still further improvements would be desirable. In general, it would be beneficial to provide further improved medical systems, devices, and methods. More specifically, it would be beneficial to provide transseptal access systems that are tailored to the capabilities and attributes of the new, balloon articulated systems so as to facilitate treatment of the mitral valve and other heart structures adjacent to the left atrium and/or left ventricle of the heart. It would be particularly helpful if these improved systems could be used to direct relatively large-profile, highly flexible prosthetic mitral valve deployment components (and the like) from the right atrium, without having to resort to the use of unnecessarily large, unnecessarily stiff, and/or otherwise excessively trauma-inducing transseptal delivery systems.

### BRIEF SUMMARY OF THE INVENTION

The present invention generally provides improved medical devices, systems, and methods. The structures described herein are particularly well suited for catheter-based structural heart therapies, including for transseptal mitral valve therapies such as those involving positioning of prosthetic mitral valves, mitral valve repair tools, and the like in alignment with target native tissues of the mitral valve of the heart. The prosthetic mitral valves can have relatively large profiles even when configured for insertion into the body, and there may be benefits to using catheter structures that can be quite laterally flexible to facilitate accurate alignment of therapeutic tools with the target tissues, with exemplary articulated systems often including articulation balloon arrays. To provide transseptal access for these large profile, highly flexible catheter tools, without unnecessarily increasing the size of the transseptal puncture, the articulated catheters can optionally be advanced over a deflectable or pre-bent, super stiff guidewire, with the bend of the guidewire extending within the right atrium so as to direct the advancing catheter laterally (and transseptally) from within a guidewire lumen of the catheter. Telescoping transseptal access systems are also provided that can make use of steering segments that are disposed proximal of a relatively rigid catheter segment supporting a prosthetic valve by engaging tissue adjacent the right atrium near the proximal end of the valve, and by telescoping a relatively rigid needle guide distally from the valve across the right atrium to engage tissue of the fossa ovalis or other target puncture site. Optional hybrid pull-wire/balloon articulation systems may employ relatively stiff pull-wire articulation within the right atrium, and relatively flexible balloon articulation systems within the left atrium. Alternative hybrid mechanical/fluid catheter systems may include pneumatic or hydraulic (or both) drive elements in a catheter base, with articulation being transmitted along the flexible catheter shaft by pull-wires or other laterally flexible mechanical movement transmitting bodies. Along with mitral valve replacement and repair, embodiments of these systems may be employed for left atrial appendage closure, intracardial ablation for treatment of atrial fibrillation and other arrhythmias, and the like.

In a first aspect, the invention provides a hybrid mechanical/fluidic catheter system for treating a patient. The system comprises a flexible catheter assembly having a proximal catheter interface and a distal portion with an axis therebetween. An actuatable feature is disposed along the distal portion and a mechanical drive member extends proximally along the axis. A driver assembly has a fluid supply and a driver interface releasably coupleable with the catheter interface. The fluid supply is operatively coupled with the driver interface such that drive fluid can articulate the catheter assembly when the catheter interface is coupled with the driver interface.

In another aspect, the invention provides a hybrid mechanical/fluidic catheter for use in a robotic catheter system for treating a patient. The robotic system includes a driver assembly having a fluid supply and a driver interface. The hybrid catheter comprises an elongate flexible catheter body having a proximal catheter interface and a distal portion with an axis therebetween. An actuatable feature is disposed along the distal portion and a mechanical drive member extends proximally along the flexible body. The fluid supply is operatively coupled with the actuatable feature by the mechanical drive member when the catheter interface is coupled with the driver interface.

A number of additional general features can be included, either alone or in combination, to enhance the functionality of the systems and methods described herein. For example, the fluid supply preferably comprises a receptacle or coupler for a sealed cannister containing a liquid/gas mixture. Vaporization of the gas within the cannister can facilitate providing inflation fluid at a pressure in a desired range without having to resort to pumps and motors. Alternative fluid supplies may include pumps with or without a reservoir, connectors or couplers for external pressurized fluid systems, or the like. The catheter or catheter assembly often comprises a catheter body having a distal catheter portion with an articulation balloon array and a plurality of lumens, each lumen being in fluid communication with an associated subset of the balloons. Alternative catheters may have different fluid-driven bodies, for example, one or more balloons coupled to a single lumen, bellows, or piston-driven systems, any of which might be used for catheter articulation, deployment of a prosthetic valve or other therapeutic tool, or the like.

Optionally, the drive member can comprise a pullwire or tubular shaft, and will often be laterally flexible and configured to transmit motion when used as a tension member, a compression member, a rotational drive shaft, or combinations thereof.

While aspects of the invention may be described herein with reference to the advantageous use of pistons within cylinder portions for driving pullwires, it should be understood that a variety of alternative fluid-driven actuators may be used instead of or together with piston/cylinder assemblies. For example, bellows, axially and/or radially expandable balloons, McKibben muscle systems, and other actuators may be substituted for some or all of the piston systems described herein. Similarly, alternative laterally flexible mechanical transmission members may be used in place of pullwires, including tubular sheaths (which may be used as tension members, compression members, or both, and/or may rotate about their axes to transmit articulation forces). The catheter interface is often disposed on a proximal housing supporting a first cylinder portion with a first piston axially movable therein. The fluid supply can be coupled with the first cylinder portion, and the drive member can be coupled with the fluid source by the first cylinder portion and the first piston so as to actuate the actuatable feature in response to pressure from the fluid supply. The driver interface often has a first fluid channel and a second fluid channel, and the catheter interface can have a first fluid channel and a second fluid channel coupled with a first side of the first piston and a second side of the piston, respectively. The first and second channels of the catheter interface can be configured for coupling with the first and second channels of the driver interface, respectively, so as to controllably drive the drive member in first and second opposed axial directions. Optionally, gas pressure is transmitted between the driver interface and the catheter interface, and a second piston is axially coupled with the first piston so that the second piston moves axially in a second cylinder portion when the first piston moves. The second cylinder can contain a liquid, and the second piston and cylinder can be configured to damp axial movement of the drive member so as to limit articulation speeds and the like. The proximal housing can contain a plurality of pistons movably disposed in a plurality of cylinders, a pair of the cylinders being axially coupled and laterally offset with the axis extending therebetween.

Optionally, movement of the first piston in the first cylinder portion induces rotational actuation of the actuatable feature about the axis of the catheter. Ideally, the catheter or catheter assembly includes a sensor coupled with the drive member so as to provide feedback to a processor of the drive assembly.

In a another aspect, the invention provides a guide system for accessing and treating a mitral valve of a patient. The system comprises an elongate catheter body having a proximal end and an articulated distal portion with an axis therebetween. A lumen extends along the axis, and a mitral valve treatment tool is supported by the catheter body distally of the articulated portion. A stiff guidewire is receivable in the lumen of the catheter body so that the tool and articulated portion are advanceable over the pre-bent guidewire. The guidewire has a proximal guidewire portion and a distal guidewire portion and is configured to define a bend therebetween so that, at rest, the distal portion extends primarily laterally relative to the proximal portion. The proximal guidewire portion and the bend can be sufficiently stiff that when the catheter body is advanced distally over the bent guidewire from adjacent the proximal end, the bent guidewire bends the articulable portion primarily laterally relative to the proximal guidewire portion.

A number of additional general features may optionally be included to further enhance utility of the structures described herein. For example, the proximal guidewire portion and bend may be relatively stiff, often having a stiffness associated with known super stiff or extra stiff guidewires, and optionally having a bending flexural stiffness of more than 50 GPa when measured using a 3-point bending test. The guidewire may be pre-bent, or may be deflectable by actuating a handle from outside the patient. The catheter body will often have a stiff catheter body portion proximal of the articulable portion. The stiff catheter body portion will often have a laterally stiffness greater than that of the guidewire along the bend so that the catheter body, when the bend is pulled proximally into the lumen along the stiff catheter body portion, reduces an angle of the bend to less than ½ a resting angle of the bend. The bent guidewire may have an autramatic soft distal portion distal of the bend, with the soft portion often forming a bend such as that of a J guidewire, a pig-tail guidewire, or the like.

Additional components may optionally be included, including a coronary guidewire for accessing a right atrium of a heart via an inferior vena cava from a femoral access site. A guide catheter may also be provided, with the guide catheter typically having a guide lumen and being advanceable over the coronary guidewire. A transseptal needle can be included for traversing the septum from within a lumen of the guide catheter. The bent guidewire can typically be directed or advanced distally within the guide lumen and transseptally through the transseptal needle or guide lumen.

Surprisingly, and despite being sufficiently flexible to be deflected laterally by the small-profile guidewire, the catheter body will often be relatively large in profile. The guidewire will often have a profile of less than 4 Fr, typically being about 3 Fr or less, and preferably being about a 0.035″ or 0.038″ diameter. In contrast, the catheter body that is deflected by this small guidewire often has a profile of about 12 Fr or more, typically being 17 Fr or more, preferably being 21 Fr or more, and optionally being from about 22 to about 29 Fr.

In another aspect, the invention provides a telescoping transseptal access system comprising an elongate catheter body having a proximal end and distal end with an axis there between. A lumen extends along the axis, and an at least semi-rigid catheter segment is disposed near the distal end (hereinafter referred to as the rigid segment). An articulatable body portion is proximal of the rigid segment, and the rigid segment has a rigid segment length. An extension catheter having an at least semi-rigid extension with an extension length corresponding to the length of the rigid segment of the catheter body is also included. A laterally flexible body portion of the extension extends proximally from the rigid extension. The flexible body portion is sufficiently flexible that the flexible body can move axially through a bend of the articulable portion, which can optionally be imposed from the proximal end. The extension is fittingly slidable in the rigid segment such that the rigid extension can telescope distally therefrom.

Optionally, the extension catheter has an extension lumen, and a needle body is also included, with the needle body slidably disposed in the extension lumen. The needle body can include a tissue penetrating distal tip, such as a sharpened curved Brockenbrough needle tip, a radiofrequency (RF) transseptal needle tip, or the like. An at least semi-rigid needle shaft can be slidably disposable in the rigid extension, and a flexible needle body portion may extend proximally of the rigid needle shaft so that distal advancement of the needle body from adjacent the proximal end can telescope the needle shaft from the extension to penetrate tissue after the articulable segment bends so as to align the rigid segment of the catheter body with a target puncture site. In some embodiments, the extension has a dilation tip tapering radially inwardly distally so as to facilitate advancing of the extension over the needle through a wall of a heart. Optionally, a dilation balloon can be disposed on the extension proximally of the dilation tip. The dilation balloon can have a small-profile configuration to facilitate transseptal insertion of the extension, and an inflated configuration about as large or even larger than a profile of the distal end of the catheter body. A proximal end of the balloon may be configured to fittingly engage a distal end of the catheter body so as to have a sufficiently smooth outer transition to facilitate axial advancement of the catheter body into the balloon-dilated wall of the heart.

For selecting a desired transseptal puncture site, the articulatable body portion may have X and Y steering such that it can be articulated in a first lateral bending orientation from outside the patient, and in a second lateral bending orientation from outside the patient, the second bending orientation being transverse to the first bending orientation. Preferably, the articulatable body portion comprises an articulation balloon array. To allow the catheter body proximally of the rigid segment (along or near the articulated portion) to brace against the tissue adjacent the right atrium (often along the ostium of the inferior vena cava (IVC)), the rigid segment length may be from about 1.5 cm to about 6 cm, typically being between about 1.75 cm and about 4 cm. The rigid extension can be configured to extend from the rigid segment to provide a maximum combined rigid length (and an associated minimum rigid overlap), the maximum combined length being in a range from about 2.57 cm to about 9 cm, typically being from about 3 and to about 7.5 cm. A deflection of the rigid extension relative to the rigid shaft will preferably remain less than about 15 degrees when the rigid extension extends from the rigid segment with the maximum rigid length and the articulation system is actuated so as to impose a maximum actuation-induced lateral load against a distal tip of the rigid extension. The needle and rigid extension can typically be axially extended with a force of more than about 200 gf from the proximal end while an articulation system of the catheter body maintains a desired articulation bend angle, such as when the needle engages a target puncture site and the catheter body proximal of the rigid segment engages tissue near the ostium of the IVC. Telescoping actuation forces may be imposed by manually inserting the extension body and/or needle body from outside the patient, or by a fluid-driven articulation system.

In another aspect, the invention provides a hybrid transseptal catheter system comprising a guide catheter body having a proximal end and a first articulatable portion with an axis therebetween. A tension member extends from the first articulatable portion toward the proximal end so as to vary a bend of the first articulatable portion from outside a patient body when the guide catheter is in use. A positioning catheter body is extendable distally from the articulatable portion of the guide catheter body. The positioning catheter body has a proximal portion supported by the guide catheter body and a distal end with a second articulatable portion therebetween. The second articulatable portion has an articulation balloon array.

Preferably, the guide body has a first stiffness and the positioning body has a second stiffness that is less than the first stiffness. The articulation balloon array provides the articulatable portion with X and Y steering such that it is configured to be articulated in a first lateral bending orientation from outside the patient, and in a second lateral bending orientation from outside the patient, the second bending orientation being transverse to the first bending orientation. The guide body has an axial lumen and a distal end with a distal guide body profile. The positioning catheter body can have a proximal portion extending through the lumen with a proximal profile, the articulatable portion having a distal profile larger than the lumen. In some embodiments, the positioning catheter body has a distal profile that is roughly the same as the distal guide body profile. The positioning catheter body can be movable axially within the lumen of the guide body, and the positioning catheter can have a receptacle for releasably receiving a prosthetic mitral valve.

### BRIEF DESCRIPTION OF THE DRAWINGS

FIG. 1 is a simplified perspective view of a medical procedure in which a physician can input commands into a catheter system so that a catheter is articulated using systems and devices described herein.

FIGS. 2A-2C schematically illustrates a catheter having a distal portion with an axial series of articulated segments supporting a prosthetic mitral valve, and show how the segments articulate so as to change the orientation and location of the valve.

FIGS. 3A-3C schematically illustrate input command movements to change the orientation and location of the valve, with the input commands corresponding to the movements of the valve so as to provide intuitive catheter control.

FIG. 4 is a partially see-through perspective view of an exemplary fluid drive manifold system for articulating a balloon array so as to control the shape of a valve delivery catheter or other elongate flexible body.

FIG. 5 is a simplified schematic illustration of components of a helical balloon assembly, showing how an extruded multi-lumen shaft can be assembled to provide fluid to laterally aligned subsets of the balloons.

FIGS. 6A-6C schematically illustrate helical balloon assemblies supported by flat springs and embedded in an elastomeric polymer matrix, and show how selective inflation of subsets of the balloons can elongate and laterally articulate the assemblies.

FIGS. 7 and 8 are cross-sections schematically illustrating a polymer dip coat supporting helical balloon assemblies with the balloons nominally inflated and fully inflated, respectively.

FIGS. 9-11 are cross-sections schematically illustrating a dip-coated helical balloon assembly having a flat spring between axially adjacent balloons in an uniflated state, a nominally inflated state, and a fully inflated state, respectively, with the dip coating comprising a soft elastomeric matrix.

FIG. 12 is a cross-section schematically illustrating yet another alternative dip-coated helical balloon assembly embedded within a relatively soft polymer matrix, with support coils disposed radially inward and outward of the balloon assemblies and dip-coated in a different, relatively hard polymer matrix.

FIGS. 13A-13E schematically illustrate frame systems having axially opposed elongation and contraction balloons for locally elongating and bending a catheter or other elongate flexible body.

FIGS. 14A-14E schematically illustrate frame systems having axially opposed elongation and contraction balloons similar to those of FIGS. 13A-13E, with the frames comprising helical structures.

FIG. 15 is a cross-section schematically illustrating an elongation-contraction frame similar to those of FIGS. 13A-14E, showing a soft elastomeric polymer matrix supporting balloon assemblies within the frames.

FIG. 16 schematically illustrates a pre-bent or deflectable super-stiff guidewire positioned transseptally for guiding a large diameter, highly flexible mitral valve therapy catheter.

FIG. 17 schematically illustrates a large diameter, highly flexible mitral valve therapy catheter that has been advanced transseptally over the pre-bent or deflectable super-stiff guidewire of FIG. 16 to deliver a mitral valve.

FIG. 18 schematically illustrating an alternative large diameter, highly flexible mitral valve therapy catheter system that has been advanced transseptally over the pre-bent or deflectable super-stiff guidewire of FIG. 16 to deliver a mitral valve, in which the catheter system comprises a hybrid catheter system including a pull-wire guide catheter and a valve positioning catheter having an articulation balloon array.

FIGS. 19A-19D schematically illustrate exploded components of the hybrid catheter system of FIG. 18.

FIGS. 20A and 20B schematically illustrate components of a telescoping transseptal system and its use for identifying a desirable transseptal access site.

FIGS. 21A-21C schematically illustrate penetration and dilation of the target transseptal access site using the components of FIGS. 20A and 20B.

FIG. 22 illustrates an interventional cardiologist performing a structural heart procedure with a hybrid fluidic/mechanical robotic catheter system having a trans-septal catheter.

FIG. 23 is a perspective view of a robotic catheter system in which a catheter is removably mounted on a driver assembly, and in which the driver assembly includes a driver encased in a sterile housing and supported by a stand.

FIGS. 24A-24C are a section view of a proximal catheter housing and associated interface structures, a sterile interface structure, and the driver and associated interface structures, respectively, showing how sterile isolation is provided while allowing drive fluid to flow between the driver and catheter, and also showing how quick-disconnect latch structures facilitate removal and replacement of disposable catheters with the reusable driver.

FIG. 25 is a perspective view of an alternative catheter having a rotatable catheter body, with a cutaway showing a rotation sensor for transmitting signals to the data processor of the driver in response to an orientation of the catheter body about the catheter axis.

FIG. 26 is a perspective view of driver assemblies having a clamp for releasably axially and rotationally affixing a guidewire relative to the stand.

FIGS. 27A-27D illustrate a series of steps that can be used in a method of preparing for and performing a trans-septal interventional procedure using the devices and systems provided herein.

FIG. 28 is a perspective view of a driver assembly with a hybrid fluidic/pull-wire catheter mounted thereon.

FIGS. 29A and 29B are a perspective view and an exploded perspective view, respectively, of the proximal portion of the hybrid fluidic/pull-wire catheter of FIG. 28.

FIGS. 30A-30C are schematics of pneumatic or hydraulic drive systems for use in the proximal housing of the hybrid catheter of FIG. 29A.

FIGS. 31A and 31B are a perspective view and a cross-section, respectively, of a proximal housing of a hybrid catheter.

FIGS. 32A and 32B are a perspective view and a cross-section, respectively, of an optional distal attachment to the housing of FIG. 31, showing components that can be used to drive the catheter (or an actuatable feature thereof) rotationally about the catheter axis.

FIGS. 33A-33C are perspective views of components of an optional proximal attachment to the housing of FIG. 31, showing components that can be used to laterally deflect an inner rotatable catheter of the catheter assembly.

### DETAILED DESCRIPTION OF THE INVENTION

The present invention generally provides fluid control devices, systems, and methods that are particularly useful for articulating catheters and other elongate flexible structures. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with multiple degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transverse to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the surface between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

The robotic systems described herein will often include an input device, a driver, and an articulated catheter or other robotic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool along a plurality of fluid channels. Optionally, the drive signals may comprise electromagnetic, optical, or other signals, preferably (although not necessarily) in combination with fluidic drive signals. Unlike many robotic systems, the robotic tool will often (though not always) have a passively flexible portion between the articulated feature (typically disposed along a distal portion of a catheter or other tool) and the driver (typically coupled to a proximal end of the catheter or tool). The system will be driven while sufficient environmental forces are imposed against the tool to impose one or more bend along this passive proximal portion, the system often being configured for use with the bend(s) resiliently deflecting an axis of the catheter or other tool by 10 degrees or more, more than 20 degrees, or even more than 45 degrees.

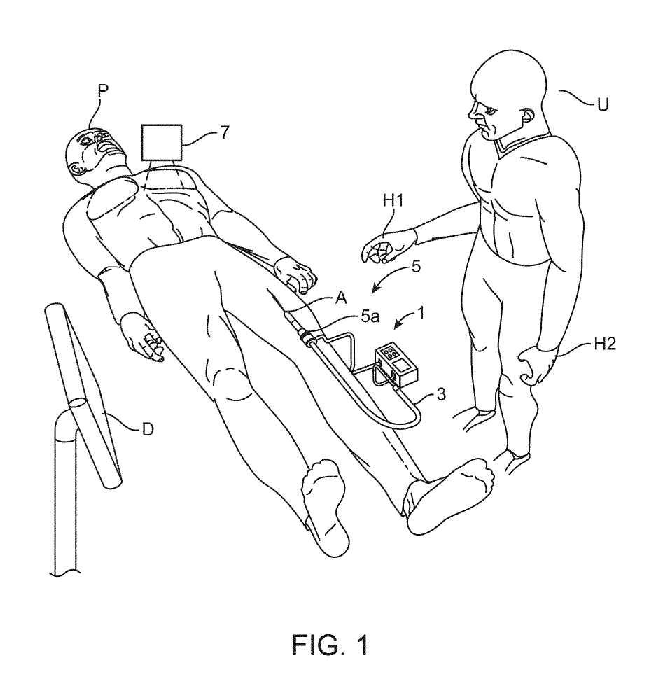

Referring first to FIG. 1, a first exemplary catheter system 1 and method for its use are shown. A physician or other system user U interacts with catheter system 1 so as to perform a therapeutic and/or diagnostic procedure on a patient P, with at least a portion of the procedure being performed by advancing a catheter 3 into a body lumen and aligning an end portion of the catheter with a target tissue of the patient. More specifically, a distal end of catheter 3 is inserted into the patient through an access site A, and is advanced through one of the lumen systems of the body (typically the vasculature network) while user U guides the catheter with reference to images of the catheter and the tissues of the body obtained by a remote imaging system.

Exemplary catheter system 1 will often be introduced into patient P through one of the major blood vessels of the leg, arm, neck, or the like. A variety of known vascular access techniques may also be used, or the system may alternatively be inserted through a body orifice or otherwise enter into any of a number of alternative body lumens. The imaging system will generally include an image capture system 7 for acquiring the remote image data and a display D for presenting images of the internal tissues and adjacent catheter system components. Suitable imaging modalities may include fluoroscopy, computed tomography, magnetic resonance imaging, ultrasonography, combinations of two or more of these, or others.

Catheter 3 may be used by user U in different modes during a single procedure. More specifically, at least a portion of the distal advancement of catheter 3 within the patient may be performed in a manual mode, with system user U manually manipulating the exposed proximal portion of the catheter relative to the patient using hands H1, H2. In addition to such a manual movement mode, catheter system 1 may also have a 3-D automated movement mode using computer controlled articulation of at least a portion of the length of catheter 3 disposed within the body of the patient to change the shape of the catheter portion, often to advance or position the distal end of the catheter. Movement of the distal end of the catheter within the body will often be provided per real-time or near real-time movement commands input by user U. Still further modes of operation of system 1 may also be implemented, including concurrent manual manipulation with automated articulation, for example, with user U manually advancing the proximal shaft through access site A while computer-controlled lateral deflections and/or changes in stiffness over a distal portion of the catheter help the distal end follow a desired path or reduce resistance to the axial movement. Additional details regarding modes of use of catheter 3 can be found in US Patent Publication No. US20160279388, entitled “Articulation Systems, Devices, and Methods for Catheters and Other Uses,” published on Sep. 29, 2016, assigned to the assignee of the subject application, the full disclosure of which is incorporated herein by reference.

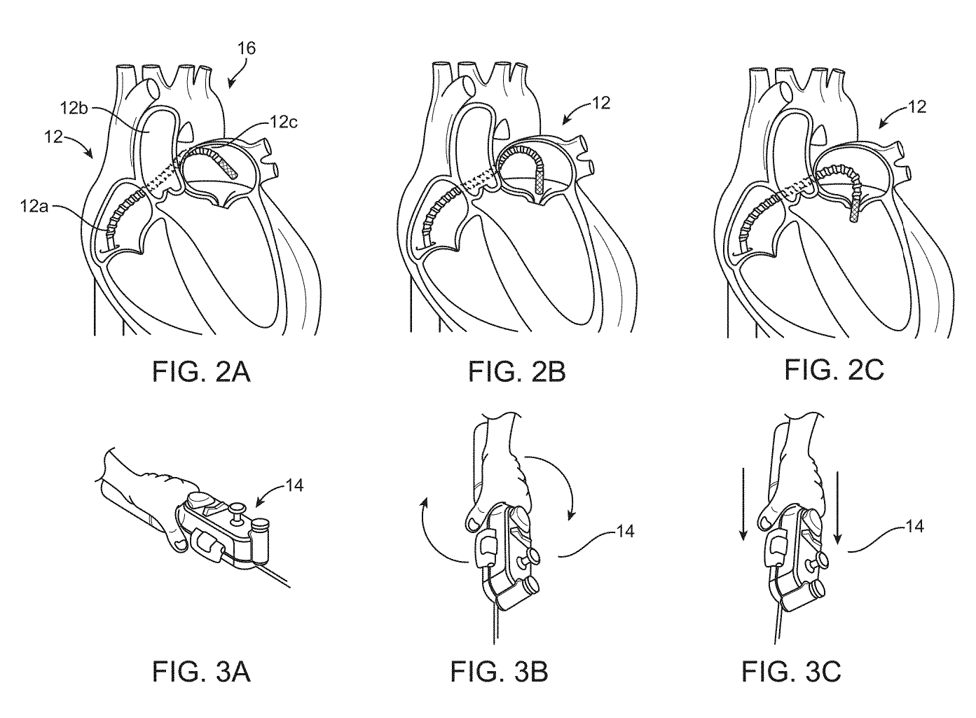

Referring now to FIGS. 2A-3C, devices and methods are shown for controlling movement of the distal end of a multi-segment articulated catheter 12 using a movement command input device 14 in a catheter system similar system 1 (described above). Multi-segment catheter 12 is shown in FIG. 2A extending within a heart 16, and more specifically with a distal portion of the catheter extending up to the heart via the inferior vena cava, with a first, proximal articulatable segment 12a bending within a right atrium of the heart toward a trans-septal access site. A second, intermediate articulatable segment 12b traverses the septum, and a third, distal articulatable segment 12c has some bend inside the left atrium of the heart 16. A tool, such as a prosthetic mitral valve, is supported by the distal segment 12c, and the tool is not in the desired position or orientation for use in the image of FIG. 2A. As shown in FIG. 3A, input device 14 is held by the hand of the user in an orientation that, very roughly, corresponds to the orientation of the tool (typically as the tool is displayed to the user in the display of the image capture system, as described above).

Referring to FIGS. 2A, 2B, 3A, and 3B, to change an orientation of the tool within the heart the user may change an orientation of input device 14, with the schematic illustration showing the input command movement comprising a movement of the housing of the overall input device. The change in orientation can be sensed by sensors supported by the input housing (with the sensors optionally comprising orientation or pose sensors similar to those of smart phones, tablets, game controllers, or the like). In response to this input, the proximal, intermediate, and distal segments 12a, 12b, and 12c of catheter 12 may all change shape so as to produce the commanded change in orientation of the tool. The changes in shapes of the segments will be calculated by a robotic processor of the catheter system, and the user may monitor the implementation of the commanded movement via the image system display. Similarly, as can be understood with reference to FIGS. 2B, 2C, 3B, and 3C, to change a position of the tool within the heart the user may translate input device 14. The commanded change in position can again be sensed and used to calculate changes in shape to the proximal, intermediate, and distal segments 12a, 12b, and 12c of catheter 12 so as to produce the commanded translation of the tool. Note that even a simple change in position or orientation (or both) will often result in changes to shape in multiple articulated segments of the catheter, particularly when the input movement command (and the resulting tool output movement) occur in three dimensional space within the patient.

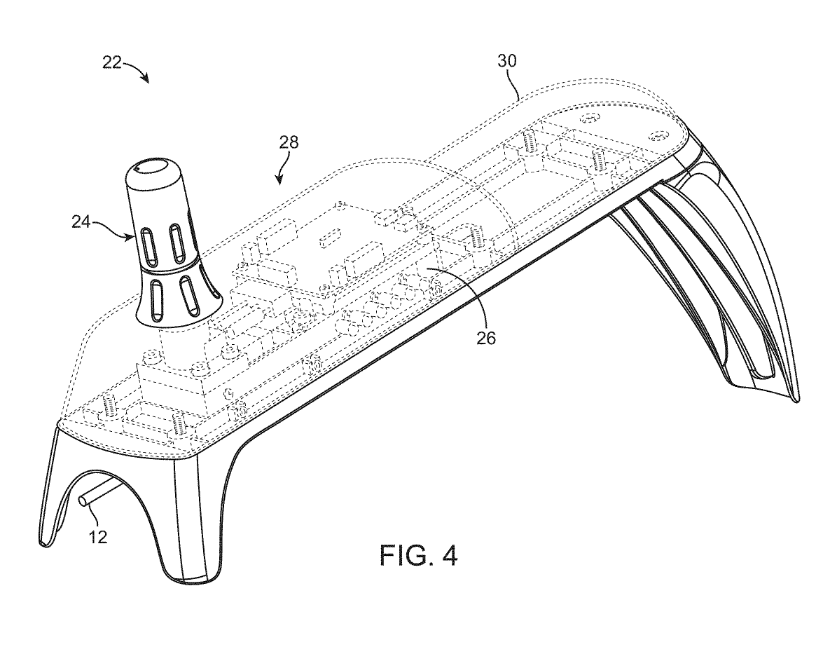

Referring to FIG. 4, an exemplary articulated catheter drive system 22 includes a pressurized fluid source 24 coupled to catheter 12 by a manifold 26. The fluid source preferably comprises a receptacle for and associated disposable canister containing a liquid/gas mixture, such as a commercially available nitrous oxide (N₂O) canister. Manifold 26 may have a series of valves and pressure sensors, and may optionally include a reservoir of a biocompatible fluid such as saline that can be maintained at pressure by gas from the canister. The valves and reservoir pressure may be controlled by a processor 28, and a housing 30 of drive system 22 may support a user interface configured for inputting of movement commands for the distal portion of the catheter, as more fully explained in co-pending U.S. patent application Ser. No. 15/369,606, entitled “INPUT AND ARTICULATION SYSTEM FOR CATHETERS AND OTHER USES,” filed on Dec. 5, 2016 (the full disclosure of which is incorporated herein by reference).

Regarding processor 28 and the other data processing components of drive system 22, it should be understood that a variety of data processing architectures may be employed. The processor, pressure or position sensors, and user interface will, taken together, typically include both data processing hardware and software, with the hardware including an input (such as a joystick or the like that is movable relative to housing 30 or some other input base in at least 2 dimensions), an output (such as a sound generator, indicator lights, and/or an image display, and one or more processor board. These components are included in a processor system capable of performing the rigid-body transformations, kinematic analysis, and matrix processing functionality associated with generating the valve commands, along with the appropriate connectors, conductors, wireless telemetry, and the like. The processing capabilities may be centralized in a single processor board, or may be distributed among the various components so that smaller volumes of higher-level data can be transmitted. The processor(s) will often include one or more memory or storage media, and the functionality used to perform the methods described herein will often include software or firmware embodied therein. The software will typically comprise machine-readable programming code or instructions embodied in non-volatile media, and may be arranged in a wide variety of alternative code architectures, varying from a single monolithic code running on a single processor to a large number of specialized subroutines being run in parallel on a number of separate processor sub-units.

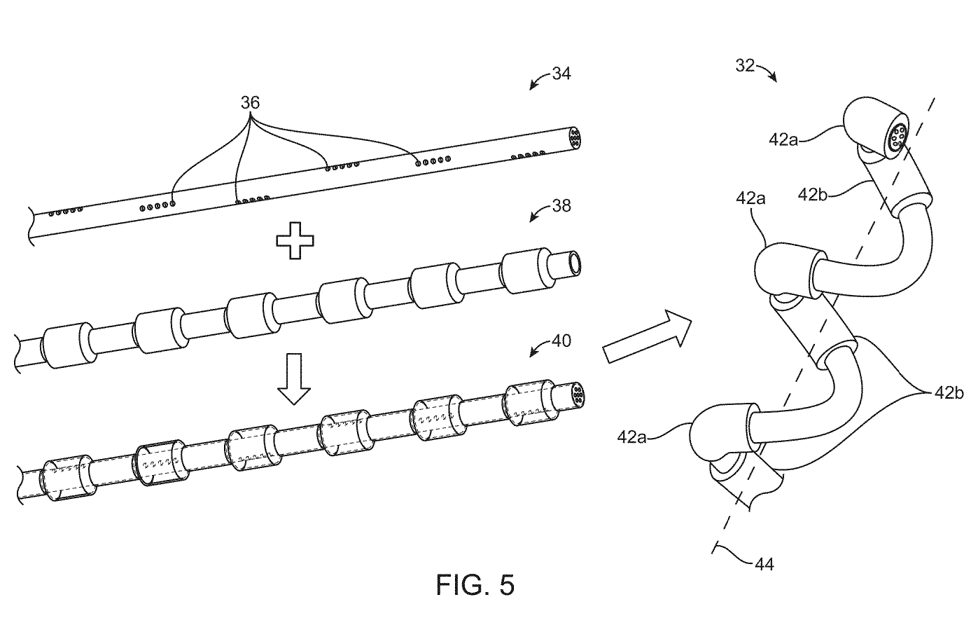

Referring now to FIG. 5, the components of, and fabrication method for production of, an exemplary balloon array assembly, sometimes referred to herein as a balloon string 32, can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using any compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

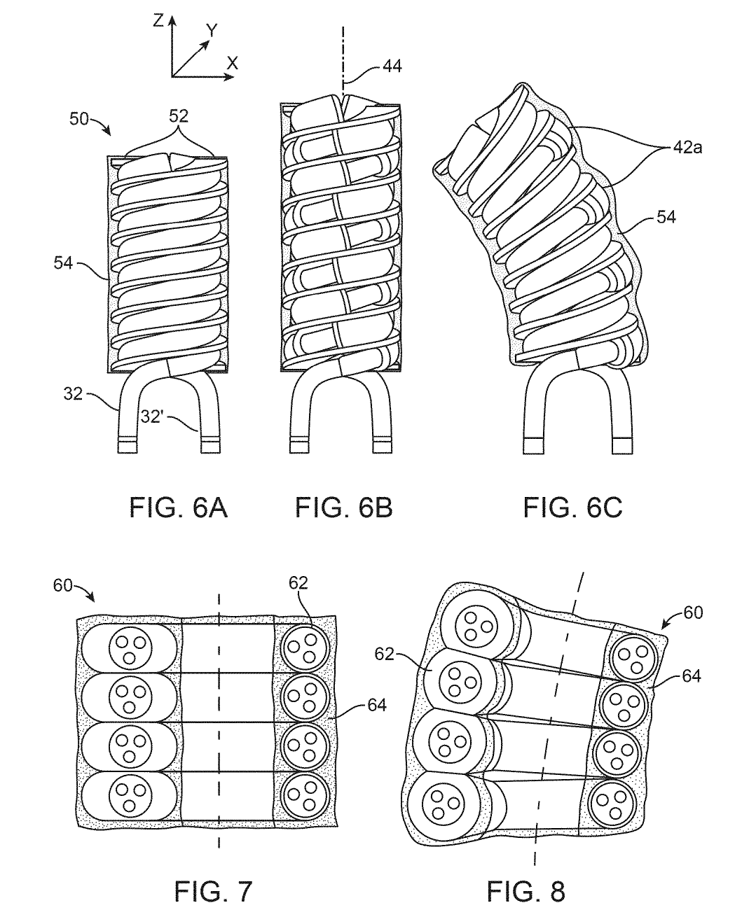

Referring now to FIGS. 6A, 6B, and 6C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 6A and 6B, inflation of subsets of the balloons surrounding the axis of segment 50 can induce axial elongation of the segment. As can be understood with reference to FIGS. 6A and 6C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 lumens), so that the total balloon array may include a series of independently articulatable segments (each having 3 or 4 dedicated lumens of one of the multi-lumen shafts, for example).

Referring still to FIGS. 6A, 6B, and 6C, articulated segment 50 includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Advantageously, matrix 54 can maintain overall alignment of the balloon array and springs within the segment despite segment articulation and bending of the segment by environmental forces.

Segment 50 may be assembled by, for example, winding springs 52 together over a mandrel and restraining the springs with open channels between the axially opposed spring surfaces. Balloon strings 32, 32′ can be wrapped over the mandrel in the open channels. The balloons may be fully inflated, partially inflated, nominally inflated (sufficiently inflated to promote engagement of the balloon wall against the opposed surfaces of the adjacent springs without driving the springs significantly wider apart than the diameter of the balloon string between balloons), deflated, or deflated with a vacuum applied to locally flatten and maintain 2 or 4 opposed outwardly protruding pleats or wings of the balloons. The balloons may be pre-folded, gently pre-formed at a moderate temperature to bias the balloons toward a desired fold pattern, or unfolded and constrained by adjacent components of the segment (such as the opposed surfaces of the springs and/or other adjacent structures) urge the balloons toward a consistent deflated shape. When in the desired configuration, the mandrel, balloon strings, and springs can then be dip-coated in a pre-cursor liquid material of polymer matrix 54, with repeated dip-coatings optionally being performed to embed the balloon strings and springs in the matrix material and provide a desired outer coating thickness. Alternatively, matrix 54 can be over-molded onto, sprayed or poured over the balloon strings and springs, or the like. The liquid material can be evened by rotating the coated assembly, by passing the assembly through an aperture, by manually troweling matrix material over the assembly, or the like. Curing of the matrix may be provided by heating (optionally while rotating about the axis), by application of light, by inclusion of a cross-linking agent in the matrix, or the like. The polymer matrix may remain quite soft in some embodiments, optionally having a Shore A durometer hardness of 2-30, typically being 3-25, and optionally being almost gel-like. Other polymer matrix materials may be somewhat harder (and optionally being used in somewhat thinner layers), having Shore A hardness durometers in a range from about 20 to 95, optionally being from about 30 to about 60. Suitable matrix materials comprise elastomeric polyurethane polymers, silicone polymers, latex polymers, polyisoprene polymers, nitrile polymers, plastisol polymers, or the like. Regardless, once the polymer matrix is in the desired configuration, the balloon strings, springs, and matrix can be removed from the mandrel. Optionally, flexible inner and/or outer sheath layers may be added.

Referring now to FIGS. 7 and 8, a simple articulated segment 60 includes a single balloon string 62 supported by a polymer matrix 64 in which the balloon string is embeeded. A multilumen shaft of balloon string 62 includes 3 lumens, and the balloons of the balloon string are shown in a nominally inflated state in FIG. 7, so that the opposed major surfaces of most of the balloons of each subset are disposed between and adjacent balloons of that subset on adjacent loops, such that pressure within the subset of balloons causes the balloons to push away from each other (see FIG. 8). Optionally, the balloons of the subset may directly engage each other across much or all of the balloon/balloon force transmission interface, particularly when the balloons are dip-coated when in the nominally inflated state. Alternatively, a layer of matrix 64 may be disposed between some portion or all of the adjacent force-transmission balloon wall surfaces of the subset, for example, if the balloon strings are dip-coated in a deflated state. As can be understood with reference to FIG. 8, inflation of one or more subsets of the balloons may separate adjacent loops of the balloon string between balloons, along the tapering balloon ends, and the like. Elastic elongation of matrix 64 may accommodate some or all of this separation, or the matrix may at least locally detach from the outer surface of the balloon string to accommodate the movement. In some embodiments, localized fracturing of the polymer matrix in areas of high elongation may help to accommodate the pressure-induced articulation, with the overall bulk and shape of the relatively soft matrix material still helping to keep the balloons of the helical balloon array in the desired alignment.

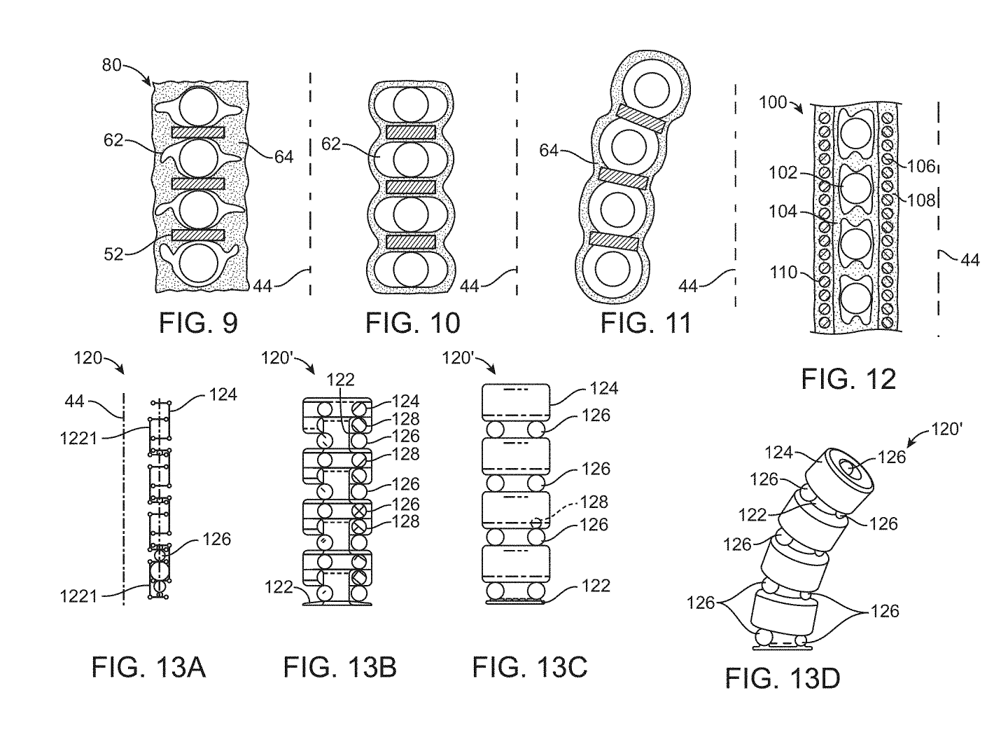

Referring now to FIGS. 9-11, an alternative segment 80 has a single balloon string 62 interleaved with a flat spring 52, and both the balloon string and spring are coated by an elastomeric polymer matrix 64. Shape setting of the balloons may be optionally be omitted, as axial compression of spring 52 can help induce at least rough organization of deflated balloons 62 (as shown in FIG. 9). Local inclusion of some matrix material 64 between the balloon walls and adjacent spring surface (see FIG. 10) may not significantly impact overall force transmission and articulation, particularly where the balloons are generally oriented with major surfaces in apposition, as the pressure force can be transmitted axially through the soft matrix material. Alternatively, the balloons may be nominally inflated during application of the matrix material, as noted above, providing a more direct balloon wall/spring interface (see FIG. 11). As with the other embodiments of segments described herein, flexible (and often axially resilient) radially inner and/or outer sheaths may be included, with the sheaths optionally comprising a coil or braid to provide radial strength and accommodate bending and local axial elongation, such inner and/or outer sheaths often providing a barrier to inhibit release of inflation fluid from the segment should a balloon string leak.

Referring now to FIG. 12, an exemplary segment 100 was fabricated with an intermediate sub-assembly including balloon string 102 embedded in an intermediate matrix 104. An inner sheath is formed radially inward of (and optionally prior to the assembly of) the intermediate sub-assembly by embedding an inner spring 106 within an inner matrix 108. An outer sheath is formed radially outward of (and optionally after assembly of) the intermediate assembly, with the outer sheath including an outer spring 110 and an outer matrix. Note that as in this embodiment, it will often be beneficial for any inner or outer spring to be counterwound relative to the balloon string. First, when the loops of the springs cross the balloons it may help inhibit radial protrusion of the balloons through the coils. Second, it may help to counteract rotational unwinding of the balloon coil structure with balloon inflation, and thereby inhibit non-planar articulation of the segment form inflation of a single balloon subset. Alternative embodiments may benefit from harder matrix materials encompassing the inner or outer springs (or both), from replacing the inner or outer springs (or both) with a braid or eliminating the springs altogether, or the like.

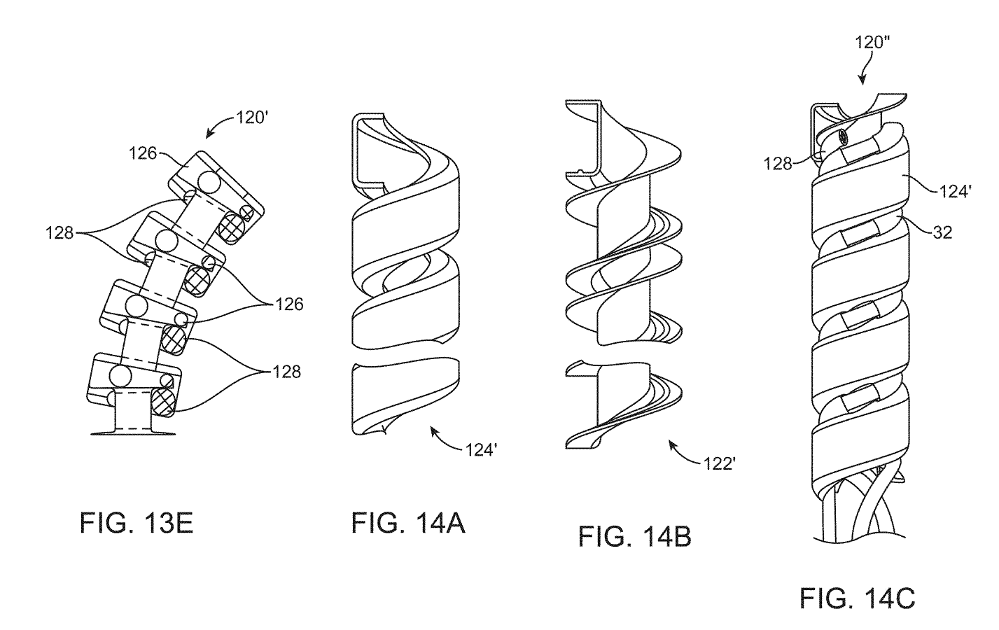

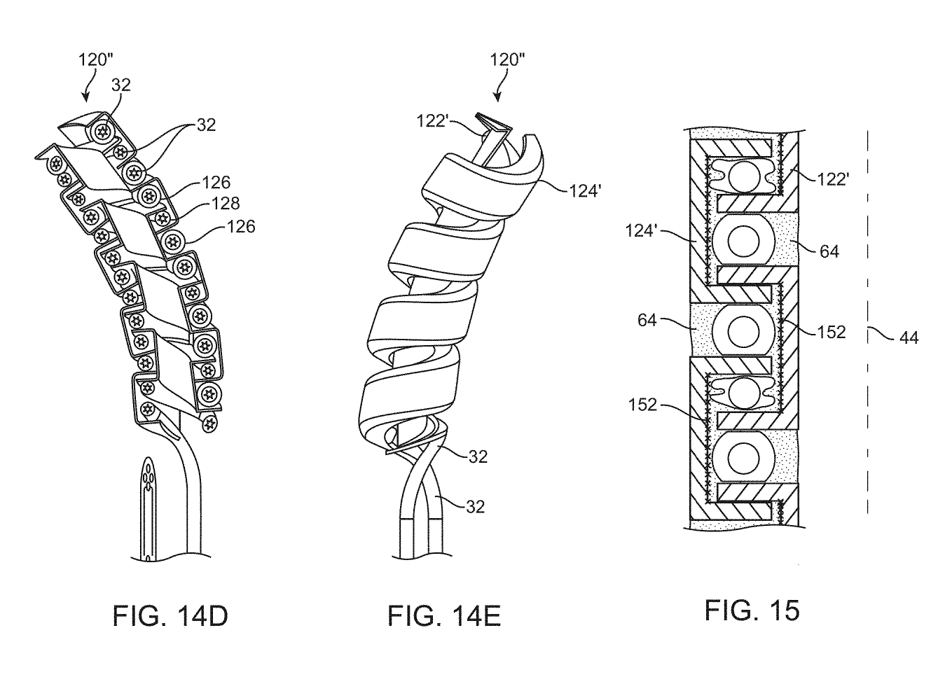

Referring now to FIGS. 13A-14E, alternative segment structures include opposed balloons disposed within channels of segment frames or skeletons to locally axially elongate or contract the frame, thereby laterally bending the frame or changing the axial length of the frame. Referring first to FIG. 13A, a schematically illustrated frame structure 120 includes an axially interleaved set of frame members, with an inner frame 122 having a radially outwardly open channel, and an outer frame 124 having a radially inwardly open channel. The channels are both axially bordered by flanges, and radially bordered (at an inner or outer border of the channel) by a wall extending along the axis. A flange of the inner frame extends into the channel of the outer frame, and a flange of the outer frame extends into the channel of the inner frame. Axial extension balloons 126 can be placed between adjacent flanges of two inner frames or between flanges of two adjacent outer frames; axial retraction balloons 128 can be placed between a flange of an inner frame and an adjacent flange of an outer frame. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), inflation of a subset of extension balloons 126 along one side of the frame locally extends the axial length of the frame and can bend the frame away from the balloons of the subset. A subset of retraction balloons 128 is mounted in opposition to that local extension, so that inflation of those retraction balloons (with concurrent deflation of the extension balloons) may move the flanges between the balloons in the opposed direction, locally decreasing the length of the frame and bending the axis of the frame toward the inflating retraction balloons. As can be understood with reference to FIGS. 13B-13E, annular frame segments 120′ may have an axially series of ring-shaped inner and outer frames defining the flanges and channels. As shown in FIGS. 14A-14E, helical versions of the frame system may have helical inner and outer frame members 122′, 124′, with extension balloons 126 and retraction balloons 128 being disposed on multiple helical balloon strings extending along the helical channels.

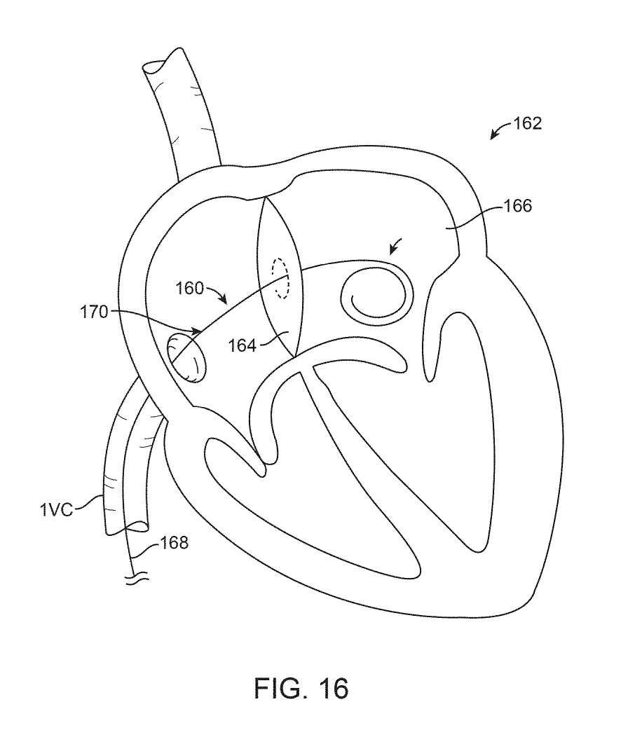

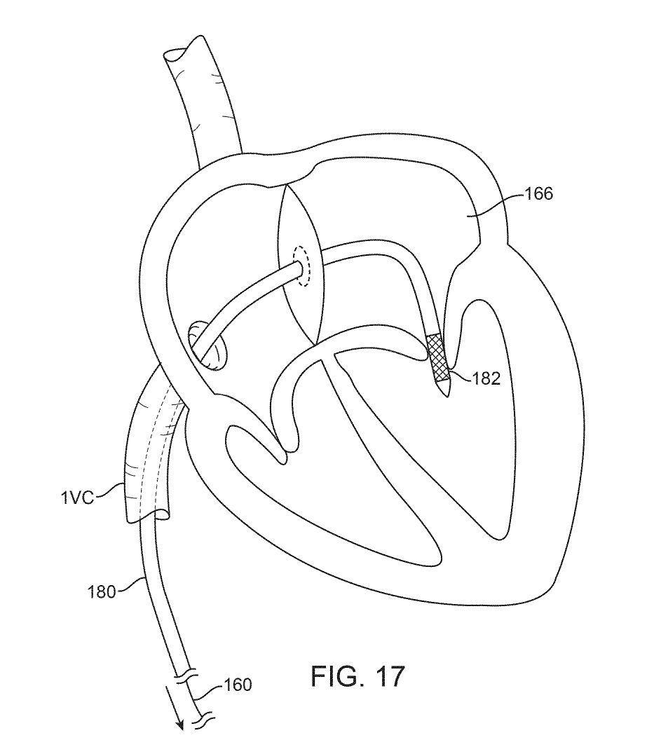

Referring now to FIG. 15, embedding the balloons within the helical frames 122′, 124′ or ring frames described herein within polymer matrix 64 may help maintain alignment of the subsets of balloons despite frame articulation. Articulation performance may be enhanced by the use of soft matrices (with Shore A durometers of 2 to 15), and by inhibiting adhesion at the frame/matrix interface 152 between the axial wall of the frames and the matrix in the channels. Preferably, a slippery interface 152 is provided by a low-friction surface in the channels of the frames between flanges, such as by coating the axial walls with a mold release agent, a PTFE polymer coating or flange material, or the like.

Referring now to FIG. 16, a pre-bent or deflectable super-stiff guidewire 160 is shown positioned transeptally in preparation for guiding of a balloon-actuated mitral valve deployment catheter. Guidewire 160 has been advanced into the heart 162 distally through an inferior vena cava IVC. Guidewire 160 extends into the right atrium through an ostium of the inferior vena cava IVC, and has been advanced through the septum 164 to the left atrium 166. Guidewire 160 may have a structure adapted from, and/or be formed by modifying, any of a number of alternative commercially available guide wires having sufficient stiffness in bending. Suitable commercial guide wires which may be bent prior to insertion into the patient to form the desired bend within the right atrium may include the Amplatz Super-Stiff and Backup Meier guide wires available from Boston Scientific, the Lunderquist™ Extra-stiff guide wires available from Cook, or the like. These known guidewires may further be modified to have shorter atraumatic distal tips with lengths of about 2 cm or less, optionally being about 1 cm or less; and optionally to have a proximal handle or fitting and a length between the proximal fitting and the atraumatic distal portion that inhibits the stiff portion extending distally beyond the catheter (so that the atraumatic tip inhibits damage to the surrounding catheter). The stiff portions of guidewire 160 may, for example, have a bending flexural stiffness of more than 40 GPa, often more than 50 GPa, optionally being more than 60 GPa, with some benefiting from more than 100 GPa when measured using a 3-point bending test. Such guidewire stiffness can be more fully understood with reference to an article available via https://www.ncbi.nlm.nih.gov/pubmed/22149229 and the 3-point bending test is more fully explained, for example, at https://en.wikipedia.org/wiki/Three_point_flexural test. Suitable guidewires will often have a profile size of between about 0.030″ and 0.045″, typically being between 0.032″ and 0.040″, and ideally being from about 0.034″ and 0.039″. Deflectable guidewires having the desired stiffness may have diameters that are within the above ranges or in ranges that extend to larger sizes, optionally being in a range from about 0.030″ to about 0.060″. Such deflectable guidewires typically have removable proximal actuation handles that can apply desired tension to a pullwire to impose an associated desired bend within the right atrium, with the angle of the bend being adjustable by the system user for that patient from outside the patient body. A number of suitable deflectable guidewire structures have been described in the patent literature and/or commercialized, and may be adapted to for use in the systems described herein, optionally by increasing component diameters and/or replacement of component materials with higher modulus metals along the bend, by shortening a length of the atraumatic flexible distal tip, and the like.

As shown in FIG. 16, a proximal portion 168 of guidewire 160 is substantially straight, and extends to a bend 170 of from about 45° to about 135°, more typically about 70° to about 120,° and ideally about 90° (+/−10°). A radius of bend 170 may be from about two to about 7 cm. Distally of bend 170, a stiff segment of guidewire 160 extends laterally by a distance in a range of from about one half to about 5 cm. Distally of the stiff lateral segment of guidewire 160, the guidewire structure transitions to an atraumatic, relatively soft bent segment, with the soft portion often biased to take, when at rest, the shape of a curve such as a circular “pig-tail”, a “J”, or the like.

Referring now to FIG. 17, a balloon articulated mitral valve deployment catheter 180 has been advanced distally over guidewire 160, with the guidewire guiding the catheter through the right atrium and septum of the heart. Optionally, the guidewire may remain in position with the articulated portion bending the axis of both the catheter and the soft end portion of the guidewire within the left atrium. Alternatively, guidewire 160 may be withdrawn proximately once the catheter 180 has been advanced so that a prosthetic valve 182 releasably mounted on catheter 180 is positioned in the left atrium 166.

The relative stiffness of valve deployment catheter 180 will often vary significantly along the axial length between the proximal end and prosthetic valve 182. The prosthetic valve and the associated structure of the catheter that supports the prosthetic valve in a small-profile configuration suitable for endovascular insertion and positioning will often be quite stiff, typically being at least semi-rigid so that it is not significantly laterally bent by the guidewire. Hence, the valve and its associated receptacle on the catheter may temporarily straighten (i.e., at least partially decrease the angle of) the bend of the guidewire as it advances distally thereover, with this rigid segment having a length in a range from about 1.75 cm to about 4 cm. The steerable portion of catheter 180 (which may have a resting length in a range from about 2.5 to about 15 cm, typically being from about 4 cm to about 12 cm) is often quite flexible to facilitate lateral bending of the catheter body via the articulation balloons (or other articulation mechanism), with this laterally flexible articulated portion typically decreasing the angle of bend 170 by less than ⅔, more often decreasing the bend by ½ or less (so that, for example, if the bend formed 90 degree when at rest, when the catheter is advanced over the bend the angle remained at 45 degrees or more). Optionally, the articulated portion of the catheter may be driven to a bent configuration when disposed over bend 170 to help maintain the bend angle. To facilitate advancing the catheter over bend 170 of the pre-bent guidewire sufficiently that the valve is far enough into the left atrium to reach the mitral valve, it will often be advantageous to also have an unarticulated flexible (in at least one lateral bending orientation) passive segment of the catheter disposed proximal of the articulated portion, with the flexible passive segment typically having a flexibility such that when bend 170 is disposed therein the angle of the bend decreases by less than ⅔ (as compared to the bend at its resting state), more often decreasing the bend by ½ or less, with the flexible passive segment often having a stiffness greater than that of the articulated segment in its resting state. The total length of the flexible articulated portion and the flexible passive segment may extend from valve 182 proximally by a distance of from about 8 to about 25 cm. To facilitate proximal withdrawal of bend 170 and the stiff lateral segment of guidewire 160 through the advanced catheter 180 and the inferior vena cava IVC for removal, the catheter body may be relatively stiff proximally of the guidewire bend when the catheter has been advanced so that the valve is positioned for deployment, with the stiff proximal portion of the catheter often decreasing an angle of bend 170 by more than ⅔ (so that, for example, a 90 degree bend would have an able of less than 30 degrees), typically by ⅚ or more, when advanced over the bend.

Referring now to FIGS. 16, 17, and 20A, it will often be advantageous to anchor the valve treatment catheters described herein locally within or adjacent the heart by bending the catheter so as to engage tissues of the heart and adjacent vasculature with the catheter sufficiently to inhibit relative motion. As a result, the distal portion of catheter 180 can move with physiological movements (such as a heartbeat and/or breathing). Optionally, bend 170 may help provide this anchoring. More specifically, when the catheter is advanced axially for valve deployment, the passive and/or articulated flexible portion of catheter 180 may extends proximally of the right atrium and into the IVC. By withdrawing bend 170 proximally into the IVC, the bend may impose an anchoring bend in catheter 180, the outer surface of the catheter engaging the luminal wall of the IVC sufficiently to inhibit movement of the catheter in at least on degree of freedom. The positioning catheter can be articulated to position the valve relative to the anchoring engagement, and when deployment is complete, the bend can then be pulled proximally into a stiff proximal segment of the catheter for removal. Accurate movement of the prosthetic valve (or other diagnostic or therapeutic tool supported by catheter 180) relative to the anchoring engagement between the catheter and IVC (or other tissues) may benefit by reversibly stiffening any passive flexible segment of the catheter disposed therebetween, with such stiffening optionally being provided by inflation of a subset of balloons disposed along the passive flexible segment, by including gooseneck assembly including an axial stack of annular bodies with rounded ends and a tension member to axially lock the assembly, or the like. In general, to provide any of the functionality described herein for delivery of a prosthetic mitral valve or other mitral valve therapies, suitable lengths of the catheter segments can be determined empirically and/or from anatomical measurement references such as https://www.researchgate.net/publication/294260728: “Anatomy of the true interatrial septum for transseptal access to the left atrium,” Article in Annals of Anatomy—Anatomischer Anzeiger⋅February 2016 DOI: 10.1016/j.aanat.2016.01.009, the disclosure of which is incorporated herein by reference.

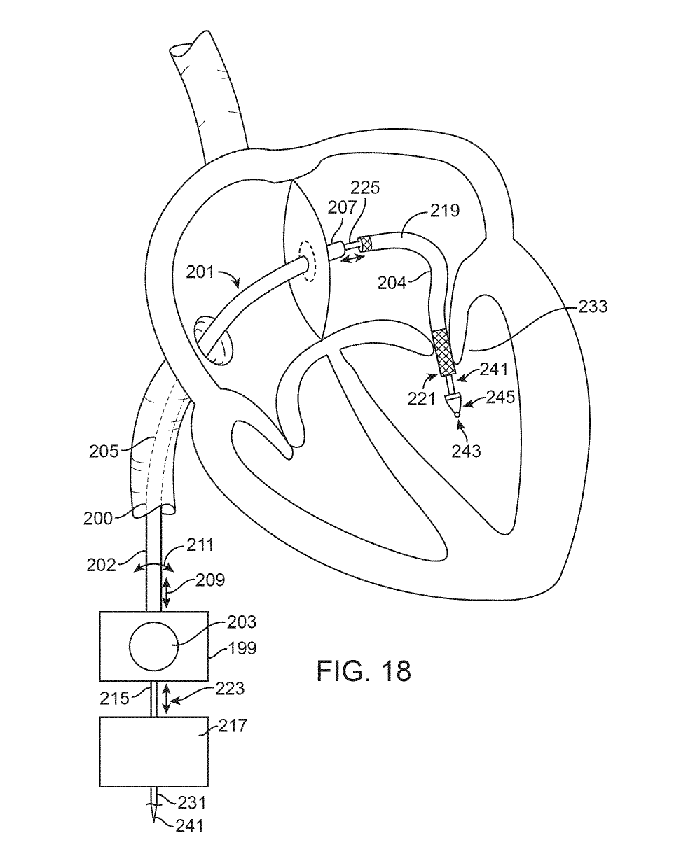

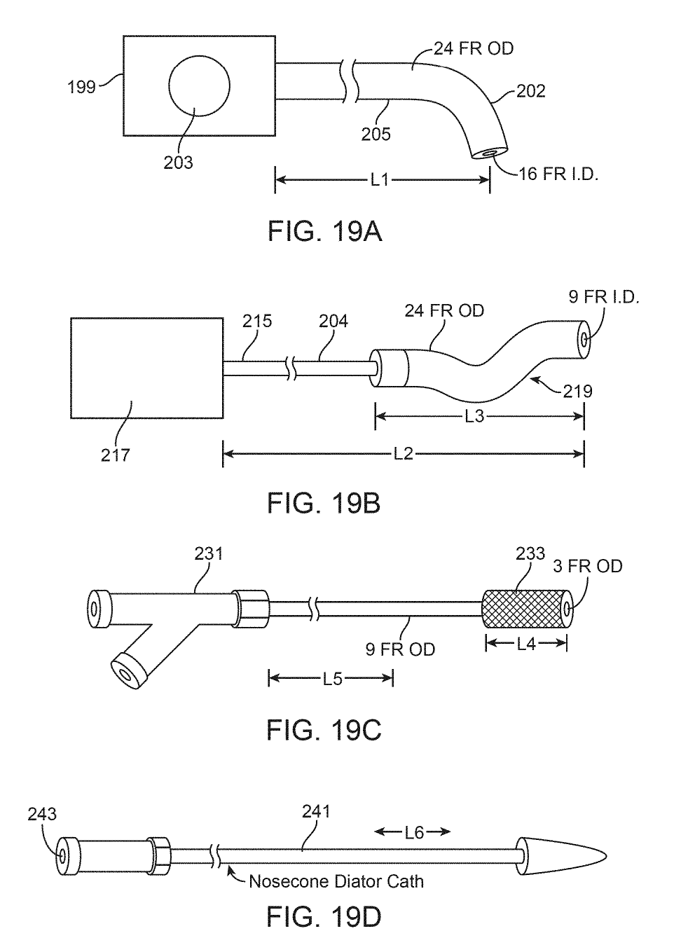

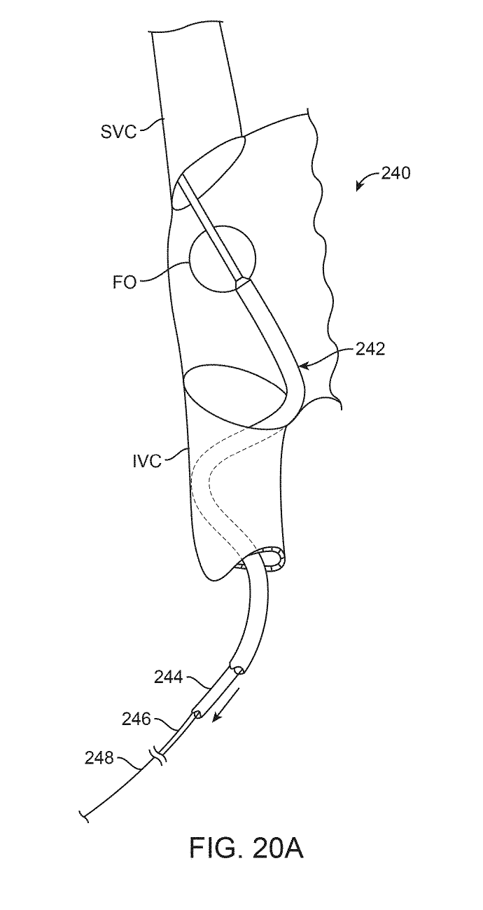

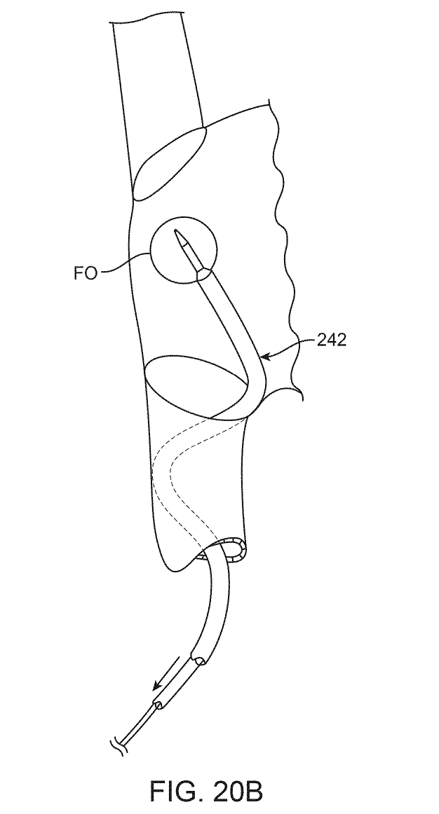

Referring now to FIGS. 18 and 19A-19D, an alternative hybrid mitral valve deployment catheter system 200 includes a pull-wire articulated guide catheter 202 and a balloon-articulated prosthetic valve therapy positioning catheter 204. Guide catheter 202 has a catheter body with a profile in a range from about 18 to about 36 Fr, typically being in a range from about 20 to about 30 Fr, and ideally having a 24 French profile, and an axial lumen which can slidably receive a catheter having a profile in a range from about 12 to about 22 Fr, typically being in a range from about 13 to about 19 Fr, and ideally for receiving about a 16 French profile catheter therein. A proximal housing 199 of the guide catheter includes an articulation knob 203 or robotically actuated mechanism that allows deflection of a distal articulated segment 201, with a pull wire extending from the proximal housing to the articulated segment suitable for imposing a bend angle of at least about 90°, often up to at least about 120°.

A catheter body 205 extends distally from proximal housing 199 to a distal end 207 (which will often have a size and length suitable to extend thru the septum and into the left atrium during use, but which may alternatively remain in the right atrium adjacent the septum). A length L1 of catheter body 205 may be in a range from about 30 to about 100 cm, preferably being in a range from about 40 to about 90 cm, and ideally being in a range from about 50 to about 75 cm. Proximal housing 199 of guide catheter 202 will often be supported so as to accommodate movement along the catheter axis 209 and rotation about the catheter axis 211, and to be restrained in a fixed axial position and rotational orientation during at least a portion of a procedure. System 200 may be configured so that axial and/or rotational movement 209, 211 can be generated by robotic drive components or by manual manipulation of system components by a hand of the system user, or both. Regardless, axial movement 209 and/or rotational movement 211 can preferably be sensed by a sensor system and associated sensor signals can be transmitted to the processor system for generation of articulation drive signals.

Referring now to FIGS. 18 and 19B, a balloon articulated valve positioning catheter 204 includes an elongate catheter body 215 extending from a proximal housing assembly 217 to and along a distal articulated portion 219 to a distal tip 221. Proximal housing assembly 217 optionally includes a proximal catheter housing and an engaged fluidic driver with a fluid supply, valve manifold, processor or controller, and the like. The catheter body adjacent the proximal fluid drive housing assembly 217 has a profile sufficiently small to pass through the lumen of the guide catheter 204, with the proximal catheter portion having a profile just under about 16 French in the exemplary embodiment. The balloon articulated portion of catheter 204 may optionally also be small enough to pass through the lumen of guide catheter 204, or may alternative have a larger profile, the distal profile often at least substantially matching the outer profile of the guide catheter. In the exemplary embodiment, the distal balloon articulated portion of the positioning catheter has a profile within a French or two of the prosthetic valve and of the guide catheter (being about 24 French in the exemplary embodiment), and the distal end of the articulated portion supports the prosthetic valve, with the valve either being mounted to the distal end of the articulated portion or to a valve deployment catheter passing through a lumen of catheter body 215. A lumen optionally (though not necessarily) extends axially through positioning catheter 204 to accommodate a guidewire (typically benefiting from a guidewire lumen diameter of at least about 0.040″ or more) or a valve therapy deployment/actuation catheter (the positioning catheter then having an ID of about 12 Fr or less, often being between about 6 and 9 Fr).

As generally described above, the articulated portion of the positioning catheter 219 may have a plurality of independently articulated segments, often having between one and four segments, preferably having two or three segments. A length L2 of catheter body 215 between housing assembly 217 and a distal end of articulated portion 219 will optionally be in a range from about 50 cm to about 120 cm, ideally being about 100 cm. A length L3 of the articulated segment 219 may be in a range from about 4 to about 8 cm. A length of catheter body 215 between housing assembly 217 and the proximal end of the articulated segment 219 will generally be at least as long as a length of the guide catheter 202 (including both guide catheter body 205 and proximal housing 199), and may optionally be longer by up to about 3 cm so as to allow the user to vary a separation 223 between the articulated catheter proximal housing assembly 217 and the guide catheter proximal housing 199. This may allow the user to vary a length of the catheter extending beyond the septum; stiffness of catheter body 215 just proximal of the articulated segment 217 along an extension portion 225 having a length slightly longer than separation 223 may be locally higher than the more proximal and/or distal portions to enhance positioning accuracy of the proximal end of the articulated segment 219.

As can be understood with reference to FIGS. 18, 19C, and 19D, a valve deployment or actuation catheter 231 may extend through the valve positioning catheter 215 so as to support and deploy the prosthetic valve 233 or another valve therapy tool. Prosthetic valve 233 may have a profile in a range from about 18 Fr to about 36 Fr, preferably from about 20 to about 30 Fr, and often being about 24 Fr, and may have a length L4 in a range from about 1 to about 5 cm when in a delivery configuration, optionally being in a range from about 1.5 cm to about 3 cm, in some cases being about 2½ cm. Deployment catheter 231 may have a length L5 in a range from being about the same as the positioning catheter (optionally including the proximal housing assembly 217) to about 5 cm longer (to allow the prosthetic tool to be advanced axially beyond the positioning catheter, either robotically or manually so as to allow tactile feedback of tissue interactions by the user), the length optionally being in a range from about 75 to about 120 cm. An OD of catheter 231 between any proximal fitting and the valve or other prosthetic tool 233 will generally be slightly less than an ID of the positioning catheter 215, often being from about 6 to about 12 Fr, optionally being about 9 Fr. Therapeutic valve tool deployment mechanisms may be included in catheter 231 (such the gripper arm and release actuation mechanisms of the MitraClip system, a balloon or fluid deployment system for radially expanding the valve, or the like. Optionally, a nosecone/dilation catheter 241 may extend through the lumen of valve deployment catheter 230. The nosecone/dilation catheter typically has an OD of about 3 Fr, a lumen 242 with an ID of less than about 0.040″ (such as about 0.038″), and a length L6 longer than that of the deployment catheter (such as being about 140 cm). An OD of the nosecone 245 may roughly match that of the valve therapy tool 232.

Referring now to FIGS. 20A and 20B, a telescoping trans-septal access and mitral valve deployment catheter system 240 includes a balloon articulated mitral valve positioning catheter 242 having a lumen that fittingly receives a needle guide or extension catheter 244. A transseptal needle 246 is, in turn, slidingly disposed in a lumen of the guide catheter 244, and a guidewire 248 can be advanced through the needle.

In FIG. 20 A, an articulated portion of the mitral valve positioning catheter 242 has been articulated to a bent configuration, inducing engagement between the catheter and an ostium of the inferior vena cava IVC. The receptacle and mitral valve prosthesis render the positioning catheter at least semi-rigid along the length of the prosthetic valve, which is disposed just distal of the articulated portion. The surface of positioning catheter 242 that engages the ostium of the inferior vena cava is disposed adjacent the distal end of the articulated portion, and/or near the proximal end of the rigid valve-receiving portion. Guide catheter 244 has a rigid distal portion with a length corresponding to a length of the rigid valve-receiving portion of positioning catheter 242, the length of the rigid portion of the guide catheter typically being within about a 1.0 cm or a ⅕ cm of the length of the rigid portion of the positioning catheter. The portion of the guide catheter 244 extending proximally from the rigid distal segment is quite laterally flexible with relatively high axial stiffness (such as by including a significant coil component, optionally with one or more relatively soft polymer layer), which allows the catheter to bend easily with articulation of the positioning catheter, but which allows the guide catheter to be accurately telescoped distally of the distal end of the positioning catheter from the proximal end of the catheter system (outside the patient). Needle 246 similarly has a relatively rigid disk portion which can reside within the rigid portions of the positioning and guide catheters, and has a laterally flexible and axially stiff proximal body to allow flexing of the positioning catheter and axial advancement of the needle.

As shown in FIGS. 20A and 20B, the positioning catheter can be articulated so as to orient the axis of the relatively rigid valve through an ostium of the superior vena cava SVC. From this configuration, the telescoped rigid portion of the guide catheter can be withdrawn proximally while the positioning catheter articulates, so that the distal end of the guide catheter slides along the interior surface of the heart. This tip motion can be monitored via imaging, a sensor disposed on the tip of the guide catheter, a pressure sensing system of the catheter drive system, and/or via the position sensing system of the catheter drive system. As the tip moves from sliding engagement along the relatively thick heart wall toward and into engagement with the surface of the thin fossa ovalis FO, the tip will drop over a ridge and engagement pressure will biefly decrease. Monitoring of several passes will allow the location, shape, and configuration of the fossa ovalis FO to be determined. Regarding determination of the configuration of the fossa ovalis FO, monitoring of the movement and engagement of the guide or extension catheter toward the FO and along the surface of the FO can be used to help characterize the FO of a particular patient as belonging to one or more of the following types: a smooth fossa ovalis, a patent foramen ovale, a right-sided septal pouch, and a net-like formation. Characteristics of these different types can be understood with reference to https://www.researchgate.net/publication/294260728: “Anatomy of the true interatrial septum for transseptal access to the left atrium,” Article in Annals of Anatomy—Anatomischer Anzeiger⋅February 2016 DOI: 10.1016/j.aanat.2016.01.009. Based on this characterization, patient suitability for a mitral valve replacement or other candidate therapy may be determined, a location of the septal access site may be selected, and/or a septal penetration tool, axial force, and/or dilation tool may be selected. Regarding suitable penetration and access tools and associated forces, additional details can be found in https://www.researchgate.net/publication/272512572: “Tissue Properties of the Fossa Ovalis as They Relate to Transseptal Punctures: A Translational Approach,” Article in Journal of Interventional Cardiology⋅February 2015 DOI: 10.1111/joic.12174. Both of the above references are incorporated herein by reference.

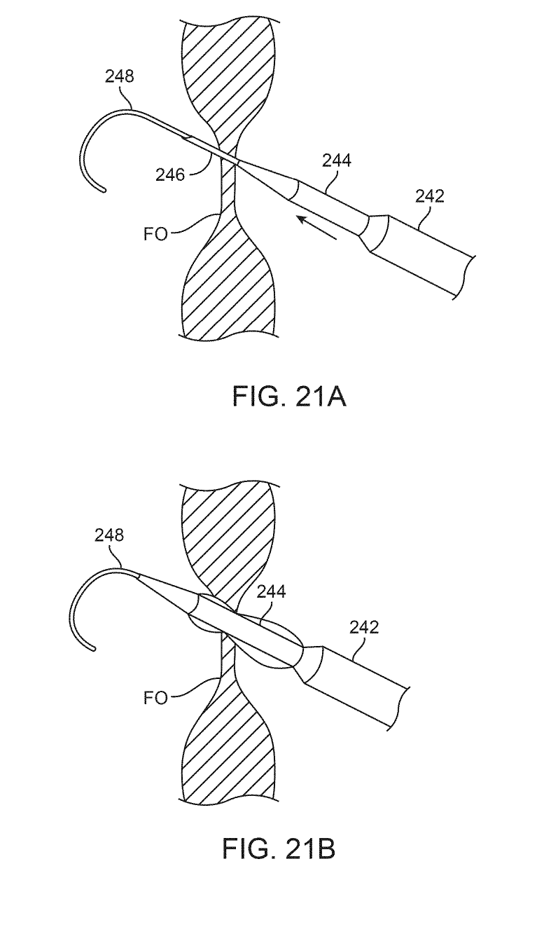

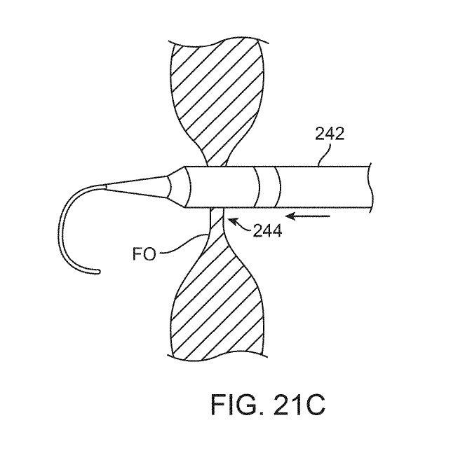

As shown in FIGS. 21A-21C, the needle and guide may be accurately oriented toward a target site along the fossa ovalis FO using the balloon articulation system, the needle guide and/or needle can engage the target site with a desired engagement force by telescoping one or both axially from the catheter, and the needle can be advanced distally through the septum while the needle is supported by the telescoped (and relatively laterally rigid) distal portions of positioning and guide catheters, and while the positioning catheter proximal of the rigid telescoped segments is braced against the heart tissue adjacent the ostium of the IVC (or another convenient location). A dilation balloon may be included in the guide catheter, with the profiles of the inflated balloon and positioning catheter corresponding so as to facilitate distal advancement of the positioning catheter into and through the septum.

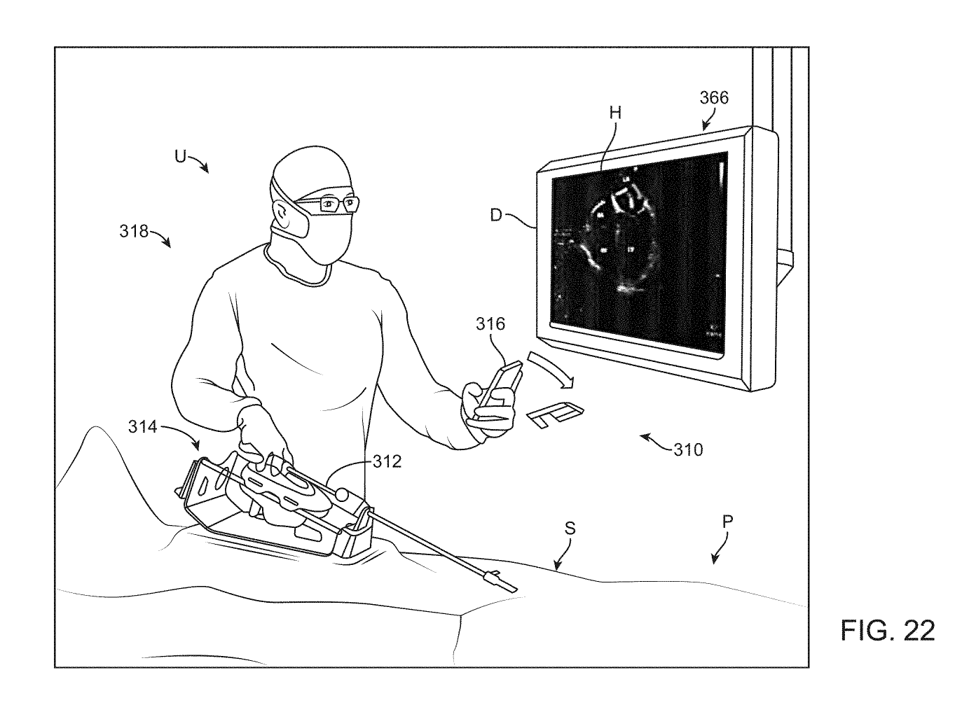

Referring now to FIG. 22, a system user U, such as an interventional cardiologist, uses an alternative robotic catheter system 310 to perform a procedure in a heart H of a patient P. System 310 generally includes an articulated catheter 312, a driver assembly 314, and an input device 316. User U controls the position and orientation of a therapeutic or diagnostic tool mounted on a distal end of catheter 312 by entering movement commands into input 316, and optionally by sliding the catheter relative to a stand of the driver assembly (and/or by manually rotating the proximal end of the catheter), while viewing a distal end of the catheter and the surrounding tissue in a display D. As will be described below, user U may manually rotate the catheter body about its axis in some embodiments.

During use, catheter 312 extends distally from driver system 314 through a vascular access site S, optionally (though not necessarily) using an introducer sheath. A sterile field 318 encompasses access site S, catheter 312, and some or all of an outer surface of driver assembly 314. Driver assembly 314 will generally include components that power automated movement of the distal end of catheter 312 within patient P, with at least a portion of the power often being transmitted along the catheter body as a hydraulic or pneumatic fluid flow. To facilitate movement of a catheter-mounted therapeutic tool per the commands of user U, system 310 will typically include data processing circuitry, often including a processor within the driver assembly as can generally understood from the description above.

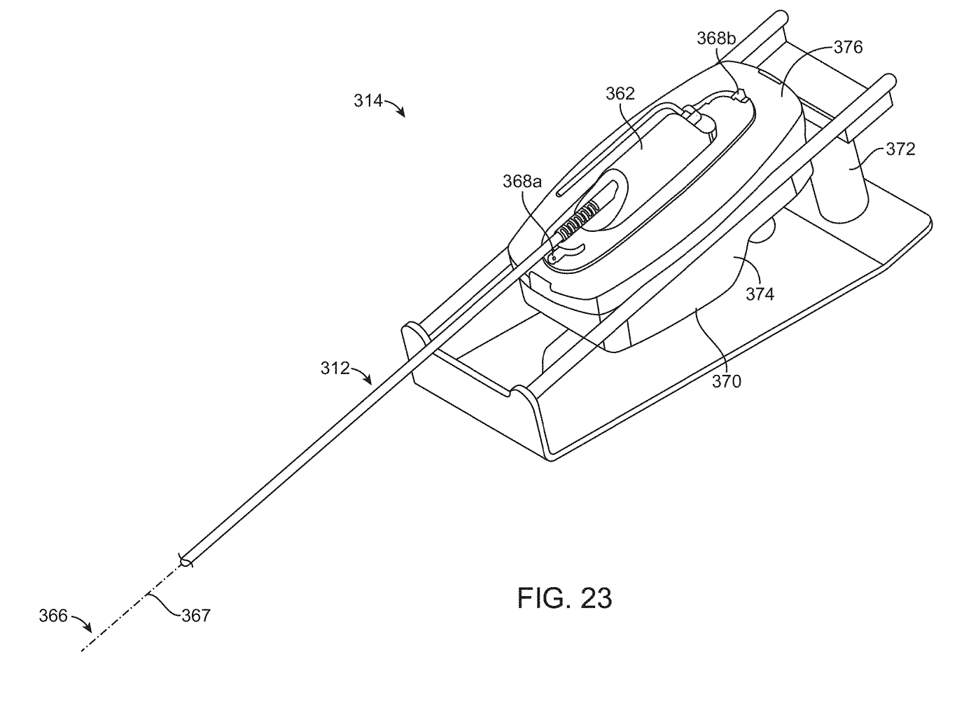

Referring now to FIG. 23, a proximal housing 362 of catheter 312 and the primary components of driver assembly 314 can be seen in more detail. Catheter 312 generally includes a catheter body 364 that extends from proximal housing 362 to an articulated distal portion 366 (see FIG. 22) along an axis 367, with the articulated distal portion optionally comprising a balloon array and the associated structures described above. Proximal housing 362 also contains first and second rotating latch receptacles 368a, 368b which allow a quick-disconnect removal and replacement of the catheter. The components of driver assembly 314 visible in FIG. 23 include a sterile housing 370 and a stand 372, with the stand supporting the sterile housing so that the sterile housing (and components of the driver assembly therein, including the driver) and catheter 312 can move axially along axis 367, preferably by sliding the sterile housing along rails of the stand. Sterile housing 370 generally includes a lower housing 374 and a sterile junction having a sterile barrier 376. Sterile junction 376 releasably latches to lower housing 374 and includes a sterile barrier body that extends between catheter 312 and the driver contained within the sterile housing. Along with components that allow articulation fluid flow to pass through the sterile fluidic junction, the sterile barrier may also include one or more electrical connectors or contacts to facilitate data and/or electrical power transmission between the catheter and driver, such as for articulation feedback sensing, manual articulations sensing, or the like. The sterile housing 370 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 372 will often comprise a metal, such as a stainless steel, aluminum, or the like for repeated sterilizing and use.

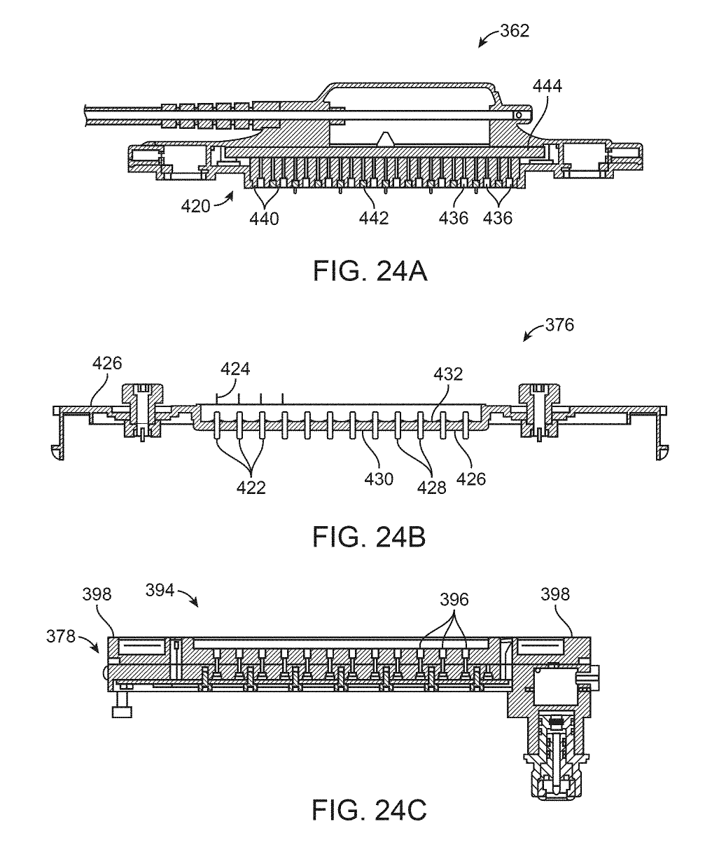

Referring now to FIGS. 24A-24C, additional structures associated with (and relationships between) the interface 394 of driver 378 and receptacle 420 of catheter housing 362 are shown. Fluid channel openings 396 of the driver interface are disposed in an array along an axis, but can be distributed in 2-dimensional patterns in other embodiments. A corresponding array of tubular bodies 422 are included in sterile junction 376, with the tubular bodies and driver channel openings 396 being aligned along parallel axes 424 that are similarly spaced. Tubular bodies 422 are supported along a plate-like region of a sterile barrier body 426 so that driver ends 428 of the tubular bodies extending from a first surface 430 of the sterile barrier body can be advanced together into channel openings 396 of the driver interface 394. The tubular bodies will often comprise a metal (such as stainless steel or aluminum) or a polymer. Opposed ends 428 of the tubular bodies adjacent a second surface 432 of sterile barrier body 426 can similarly be advanced in unison into fluid channel openings 436 of catheter interface 420. Optionally, both ends of the tubular bodies include a compliant surface for sealing against the surrounding fluid channel openings, such as by including O-rings, molding or over-molding the tubular bodies with elastomeric materials, or the like. Alternatively, tubular bodies might be associated with the driver interface or the catheter interface, or both, with corresponding receptacles on adjacent sides of the first surface 430 and second surface 432 of the sterile coupler, or any combination of the above.

To accommodate any separation distance or angular mismatch between the fluid channel openings 396, 436 and tubular bodies 422, the sterile barrier body may support the tubular bodies so as to allow them to float within a tolerance range, for example, by over-molding a softer material of the sterile barrier body 426 over a more rigid material of the tubular bodies or the like. Preferably, the tubular bodies extend through oversized apertures through the sterile barrier body 426, with radially protruding split-rings or flanges attached to the tubular bodies adjacent the opposed surfaces 130, 132 capturing the sterile barrier body but allowing the tubular bodies to slide laterally and/or rotate angularly within the apertures. In a somewhat analogous arrangement, channel openings 436 of catheter interface 120 may float laterally by forming each opening in a separate body or puck 440. The orientation and general position of the catheter channel openings can be maintained by capturing flat surfaces of pucks 440 between a first wall 442 and a second wall 444 of the catheter interface, allowing the pucks to slide laterally within a tolerance range to accommodate spacing of the tubular bodies when the opposed ends extend into the channel openings 396 of the driver interface 394. Apertures through first wall 442 may accommodate the tubular bodies to facilitate coupling, or pucks 440 surrounding openings 436 may extend through the apertures (a protruding portion of the puck being smaller than the aperture to accommodate the axial float tolerance). Note that the ends 422 of the tubular bodies and/or the channel openings 396, 436 may be chamfered to facilitate engagement, and a series of flexible polymer tubes may be bonded or otherwise affixed to the pucks 440, with the tubes extending into the catheter body or otherwise providing fluid communication between the catheter interface and balloon array.

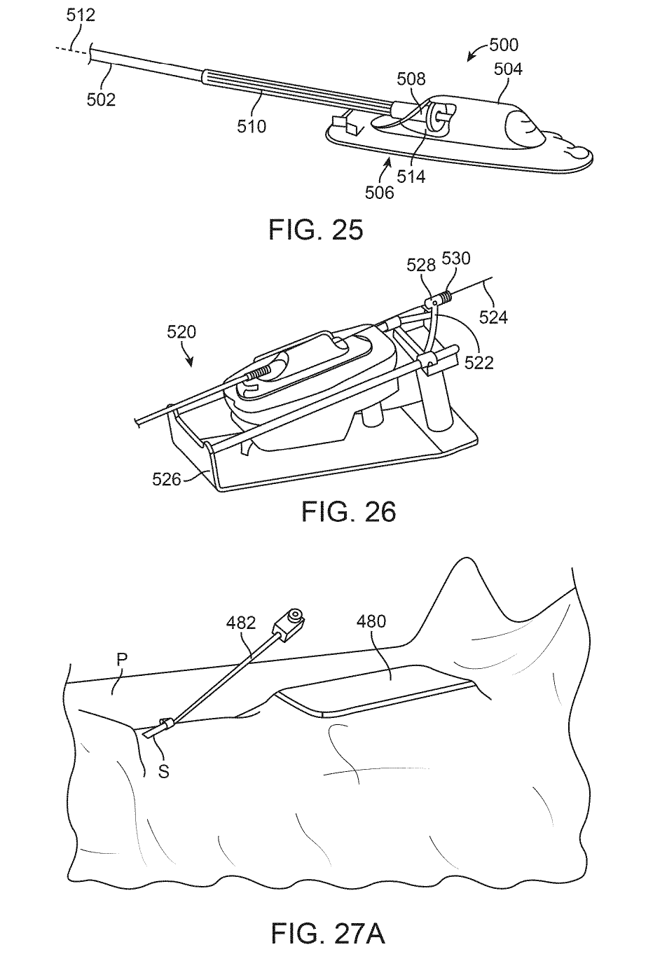

Referring now to FIG. 25, a rotatable shaft catheter 500 shares many of the structures of the catheters described above, including a catheter body 502 extending distally from a proximal catheter housing 504 having a catheter receptacle 506 configured for coupling with a driver. Catheter body 502, however, is rotationally attached to housing 504 by a rotational bearing 508 that optionally allows the user to manually rotate the catheter body about the catheter axis. Alternatively, a rotational drive mechanism (as described below) can induce rotation of the catheter relative to the housing. In manually rotatable embodiments, a handle 510 is mounted to the catheter body near bearing 508. The handle is configured to be grasped by the hand of the user and rotated about axis 512. In manual or rotationally driven embodiments, a sensor 514 senses the rotational state of the catheter and transmits catheter rotation signals to the processor of the driver, optionally via conductors of the sterile junction. Sensor 514 may comprise an optical encoder, a potentiometer, or the like. The signals will be suitable for providing real-time feedback on the catheter rotational state to the processor so as to allow the processor to calculate articulation drive signals for the articulated portion of the catheter. Note that a wide variety of alternative rotational or axial sensors may be provided, either sensing positional relationships adjacent the driver, along a length of the catheter assemblies, or the like. In some embodiments, the rotation (or axial offset) may be measured distally of housing 504, such as using an encoder or resistor affixed to a distal portion of a guide catheter surrounding catheter body 502 adjacent the articulated portion, and an optical sensing surface or electrical contact mounted to the catheter body.

Referring now to FIG. 26, an alternative driver assembly 520 has a guidewire support 522 to axially and/or rotationally affix a guidewire 524 relative to a stand 526. Guidewire support 522 has a lateral opening 528 to receive guidewire 524 laterally (relative to the axis of the guidewire) into jaws of the support. A guidewire rotational knob 530 may be affixed rotationally to the guidewire by a set screw or the like, In methods that avoid the use of a guide catheter, a guidewire (such as a super stiff guidewire or extra stiff guidewire) may instead be affixed to guidewire support 522 of the stand proximally of the driver, typically after catheter 212 is loaded retrograde onto the guidewire and has been advanced so that a distal end of the catheter is adjacent the target tissue (and so that the proximal housing of the catheter is distal of the proximal guidewire support or clamp). The stand may include both a distal releasable clamp or support for the guide catheter (as shown above) and a releasable proximal clamp or support 522 for the guidewire 524 proximal of the rails. Both the guide catheter clamp and guidewire clamp may be used together for some procedures, with the guidewire often ending proximally of (or having only a highly flexible distal portion extending into) the articulated portion of the catheter, which will often extend distally of (or be articulated distally of) the distal end of the guide catheter.

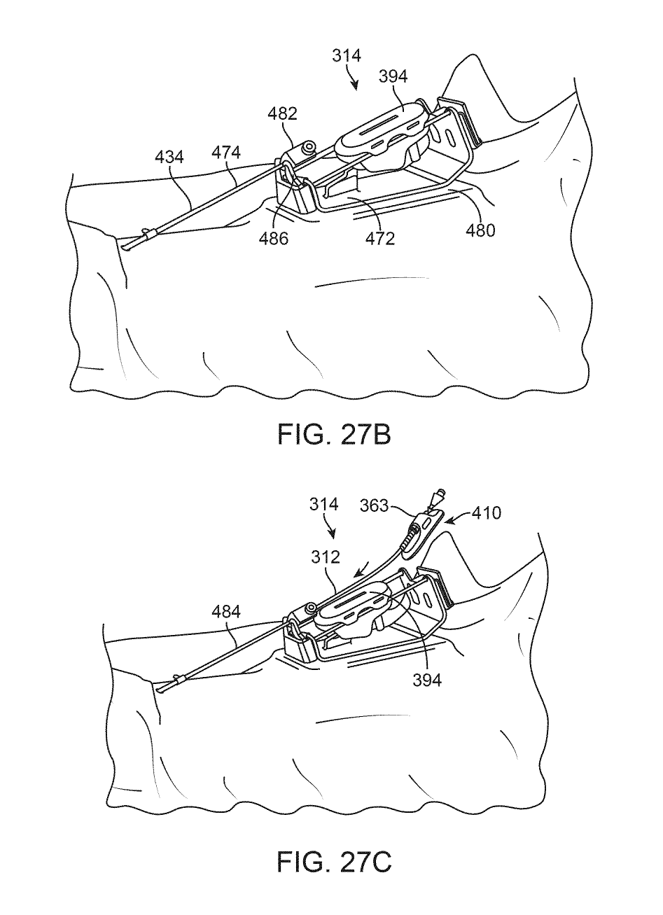

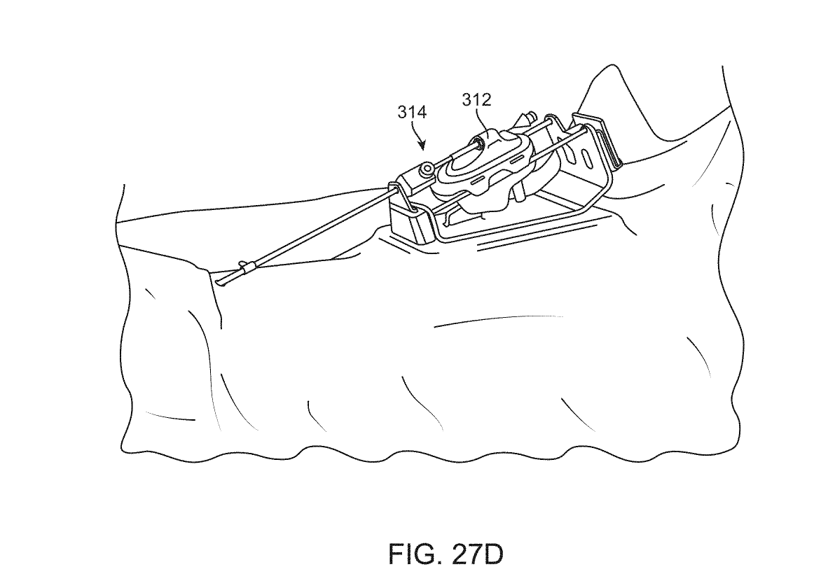

Referring now to FIGS. 22 and 27A-27D, a method for preparing robotic system 310 for use can be understood. As seen in FIG. 27A, a horizontal support surface 480 has been positioned adjacent a surgical access site S, with the exemplary support surface comprising a small stand that can be placed over a leg of the patient P (with the legs of the stand straddling a leg of the patient). A guide catheter 482 is introduced into and advanced within the vasculature of the patient, optionally through an introducer sheath (though no introducer sheath may be used in alternate embodiments). Guide catheter 482 may optionally have a single pull-wire for articulation of a distal portion of the guide catheter, similar to the guide catheter used with the MitraClip™ mitral valve therapy system as commercially available from Abbott. While a manual knob may be used to articulate guide catheter 482, and/or a fluidic drive system of the catheter and/or driver (such as those described below) may optionally be used to apply forces to the guide wire of the guide catheter. Alternatively, the guide catheter may be an unarticulated tubular structure, or use of the guide catheter may be avoided. Regardless, when used the guide catheter will often be advanced manually by the user toward a surgical site over a guidewire using conventional techniques, with the guide catheter often being advanced up the inferior vena cava (IVC) to the right atrium, and optionally through the septum into the left atrium.

As can be understood with reference to FIGS. 22, 27A, and 27B, driver assembly 314 may be placed on support surface 480, and the driver assembly may be slid along the support surface roughly into alignment with the guide catheter 482. A proximal housing of guide catheter 482 and/or an adjacent tubular guide catheter body can be releasably affixed to a catheter support 486 of stand 372, with the support typically allowing rotation and/or axial sliding of the guide catheter prior to full affixation (such as by tightening a clamp of the support).

As can be understood with reference to FIGS. 22, 27B, and 27C, catheter 312 can be advanced distally through guide catheter 482, with the user manually manipulating the catheter by grasping the catheter body and/or proximal housing 368. Note that the manipulation and advancement of the access wire, guide catheter, and catheter to this point may be performed manually so as to provide the user with the full benefit of tactile feedback and the like. As can be further understood with reference to FIGS. 22, 27C, and 27D, as the distal end of catheter 312 extends near, to, or from a distal end of the guide catheter into the treatment area adjacent the target tissue (such as into the left atrium) by a desired amount, the user can manually bring the catheter interface down into engagement with the driver interface, preferably latching the catheter to the driver through the sterile junction as described above.

In methods that avoid the use of a guide catheter such as that shown affixed to a distal clamp of the stand by support 486, a guidewire (such as a super stiff guidewire or extra stiff guidewire) may instead be affixed to a guidewire support of the stand proximally of driver assembly 314, typically after catheter 312 is loaded retrograde onto the guidewire and is advanced over the guidewire to so that a distal end of the catheter is adjacent the target tissue (and so that the proximal housing of the catheter is distal of the proximal guidewire support or clamp). The stand may include both a distal releasable clamp or support 486 for the guide catheter (as shown) and a releasable proximal clamp or support for the guidewire proximal of the rails (not shown). Both the guide catheter clamp and guidewire clamp may be used together for some procedures, with the guidewire often ending proximally of (or having only a highly flexible distal portion extending into) the articulated portion of the catheter, which will often extend distally of (or be articulated distally of) the distal end of the guide catheter.

Referring now to FIGS. 22 and 27D, the driver and sterile housing will typically be in a relatively proximal axial position relative to the stand when the catheter engages the driver, so that the user can make use of the robotic articulation of the distal portion of the catheter during final advancement of the therapeutic tool of the catheter into alignment with the target tissue. Stand 372 may optionally have a holder for input 316. In some embodiments, the input may be used to enter articulation commands while supported by stand 372. The input can optionally be affixed to the stand or the sterile housing, or mounted to the driver and manipulatable by the user through a membrane of the sterile housing, or placed on support surface 480, or the like. The user may optionally perform a portion of the final distal advancement by sliding driver assembly 314 and catheter 312 along the rails of stand 372 either manually or using a proximal fluidic drive system such as those described below, with the processor deriving articulation commands for the distal articulated portion at least in part in response to signals from an axial position sensor. Optionally, at least a portion of the final advancement of the tool of the catheter may be performed by robotically articulating the catheter.

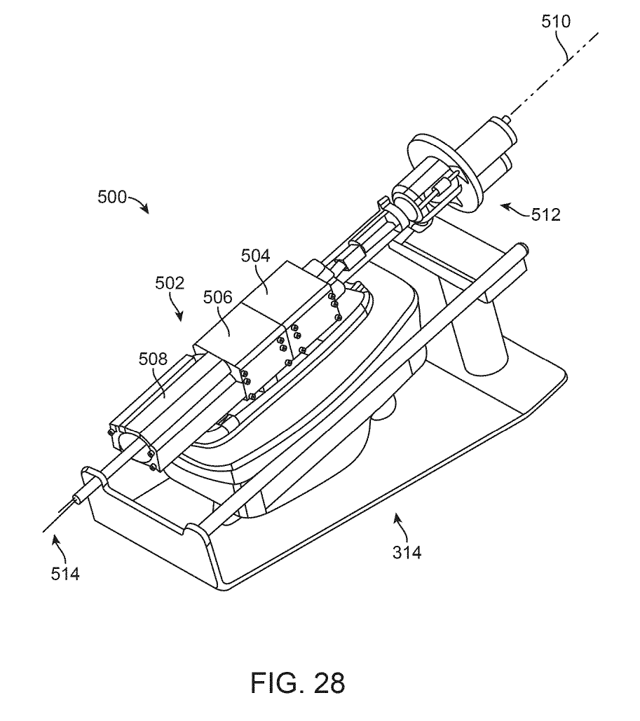

Referring now to FIG. 28, a hybrid fluidic/mechanical catheter system 500 includes a hybrid catheter 502 removably mounted to driver assembly 314. Note that hybrid system 500 can thus be used interchangeably with many of the systems described above by removal and replacement of the catheter mounted to the driver assembly, providing the benefits of robotic coordinated motion of the differing articulation degrees of freedom of the different catheters when desired. The processor of the driver included in the driver assembly will often be configured for the mounted catheter using electrical signals transmitted between the driver and circuitry of the catheter, effectively functioning as a plug-and-play system. A proximal housing 504 of hybrid catheter 502 has a catheter receptacle 420 for transmitting a plurality of drive fluid channels and a plurality of electrical signal channels.

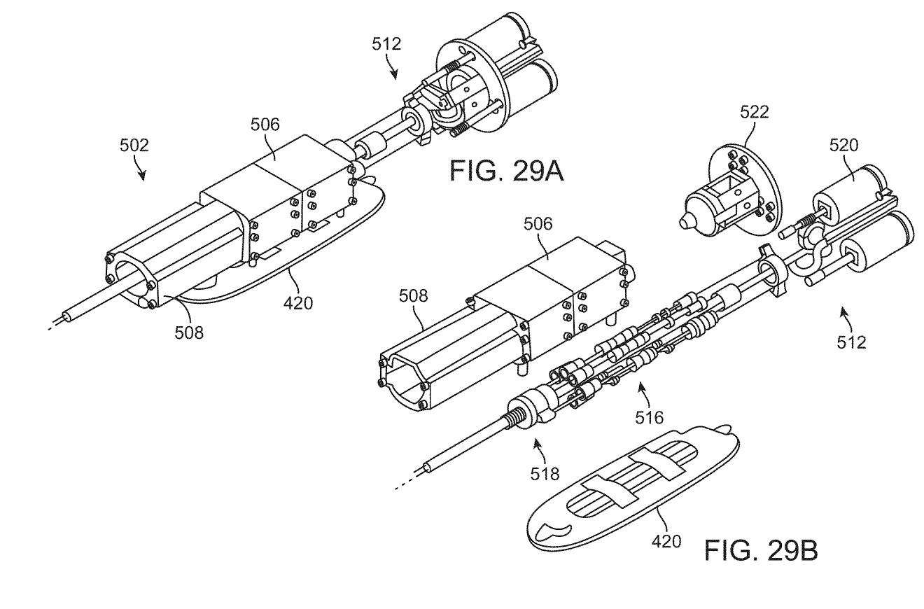

Referring now to FIGS. 28, 29A, and 29B, housing 504 generally includes a piston drive portion 506 (to convert fluid drive flows from the driver to axial mechanical motion), and may also optionally include a rotational drive portion 508 (to convert axial motion to rotational motion about an axis 510 of catheter 502) and/or an electromechanical pull-wire portion 512 (to convert electrical drive signals from the driver to axial motion of one or more pull-wires 514). A plurality of pistons is driven axially within associated cylinders of piston drive portion 506 by opposed gas pressure channels, with the axial movement damped by hydraulic dampers. Two of the piston/cylinder assemblies can be used to set relative axial positions of a pair of slide members 518 within rotational drive portion 508, and changes in those relative axial positions can be used to induce rotation of a tubular shaft or the like of the catheter system via axial threads. In electromechanical portion 512, motors 520 mounted to a rotatable carrier 522 can tension pullwires, and the carrier may also be axially positioned by another piston/cylinder assembly of drive portion 506. A wide range of alternative arrangements may also be provided, including using different combinations of the components of the hybrid catheter proximal housing portions and/or using different pneumatic, hydraulic, mechanical, and/or electromechanical components.

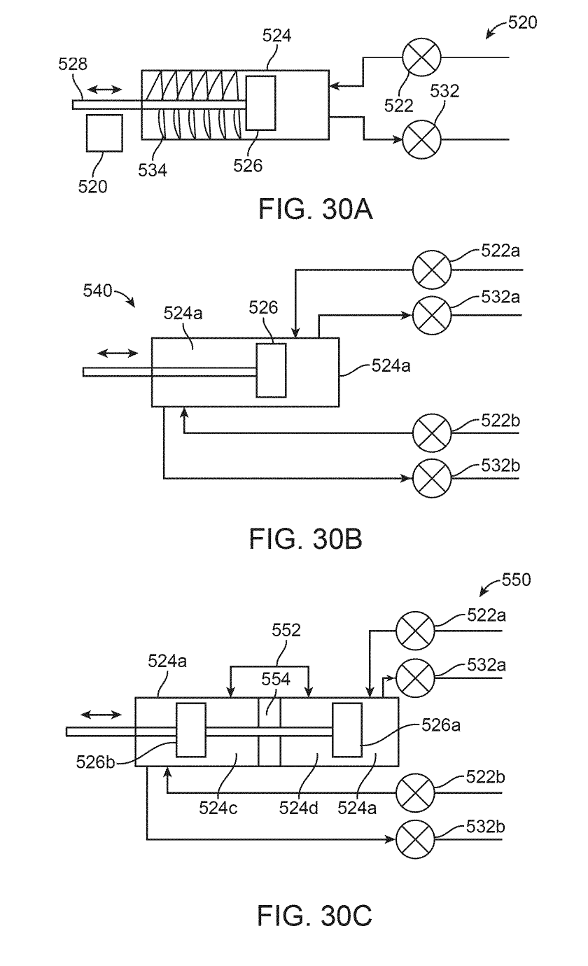

Referring now to FIGS. 30A-30C, simplified fluidic schematics of components are shown that use pistons to transform fluid flows (typically pneumatic or hydraulic) to mechanical movement (typically of a pullwire, a nested catheter or sheath, or tension/compression shaft) to help drive a particular channel or robotic degree of freedom of the catheter. As shown in FIG. 30A, a single-channel system 520 makes use of pressurized fluid from a fluid supply as regulated by a fill valve 522. Supply fluid is directed from fill valve 522 into a cylinder 524 so that the pressurized fluid can axially displace a piston 526 within the cylinder. Piston 526, in turn, axially moves a shaft 528, and the axial displacement will often be measured by a displacement sensor 530, which may be coupled to piston, shaft, a pullwire affixed to the shaft, or the like. A drain valve 532 allows inflation fluid from cylinder 524 to be released to a drain channel, the drain fluid often be released to ambient (if a benign gas is used) or being collected in a drain reservoir (for liquids). A bias spring 534 or other mechanism may oppose fluid pressure within the cylinder to allow the system to controllably move shaft 528 proximally and distally.

A number of variations of single-channel system 520 may be employed to provide desired functionality. For example, when desired (for example, to tension a pullwire to resiliently deflect a catheter shaft), the pullwire may be directly attached to piston 526, the resilient catheter structure can be used with or in place of spring 534 to oppose proximal movement of the piston, and/or the fill and drain channels may be coupled to cylinder 524 distally of piston 524 (rather than proximally as shown). The use of incompressible inflation fluids (water, saline, hydraulic fluids, etc.) may have advantages when more precise positioning of piston 526 (and hence more precise articulation at the distal portion of the catheter) are desired, and compressible inflation fluids (air, N2O, CO2, N2, etc.) may facilitate providing atraumatic tissue engagement and the use of a stable-pressure sources such as a sealed container having a gas/fluid mixture. The fill and drain valves 522, 532 may be included in the catheter housing, the driver, or a separate structure, the channels may be combined into a single fill-drain channel on the piston side of the valves (so that, for example, only a single channel of the catheter/driver interface is used to drive shaft 528), and a wide variety of sensors (including optical sensors, electro-mechanical sensors such as potentiometers or hall effect sensors), valves (including open/closed valves, proportional valves, solenoid valves, piezoelectric valves, combining the fill and drain valves into a single 3-way valve, and the like), piston seals, cylinder arrangements, and the like may be provided. Where some channels are being driven by gas and other channels are being driven by liquids, gas pressure may be used to pressurize a reservoir of liquid within the catheter housing or driver, or a micro-hydraulic motor or other fluid pressure source may be included in the catheter housing, the driver, or a dedicated separate structure; for recirculating hydraulic systems, a drain reservoir may be provided in the same or a different structure. Similar (and other) variations may be provided for each of the fluidic/mechanical piston drive transmission systems described herein.

Referring now to FIG. 30B, a two-channel opposed piston system includes two fill valves 522a, 522b and two drain valves 532a, 532b used to direct fluid along separate channels to first and second cylinder portions 524a, 524b. As illustrated, the separate inflation fluid flows urge piston 526 in opposed axial directions. Note that the supply fluid directed to the fill valves (before flowing toward the cylinder portions) will often come from a common source and be at a common pressure, and the drain fluid flowing away from the drain valves (and the cylinder portions) may be directed to a common reservoir or release port. The cylinder portions may optionally be in separate, axially off-set housings (with separate pistons being connected by a shaft or the like). When compressible fluids are used, dependent control of the opposed pressures may be advantages. For example, the axial stiffness of the shaft positioning may be varied, for example, by increasing the gas pressure on both sides of the piston to increase axial stiffness and positioning accuracy, and may be decreased by decreasing both pressures to limit tissue engagement forces and associated trauma. Hydraulic fluids can be used on both sides of the piston to provide significant stiffness against un-commanded movement in both the proximal and distal orientations, with some or all of the valves optionally being on a single three-position spool (move shaft proximally, fixed axial position, and move shaft distally).

Referring now to FIG. 30C, a damped two-channel system 550 includes many of the components described above regarding two-channel system 540, with the drain and fill valves 522, 532 controlling fluid in opposed cylinder portions 524a, 524b to urge piston portions 526a, 526b distally and proximally. Additionally, fluid is contained in a second pair of opposed cylinder portions 524c, 524d, so that axial movement of the pistons 526 increases pressure in one and decreases pressure in the other. A restricted flow path 552 allows fluid to flow at a limited flow rate between the second pair of opposed piston portions, thereby serving as a damper to limit a speed of axial movement of the output shaft. Damped two-channel system may have advantages for use of pneumatic fluids to drive the shaft, particularly when non-compressible fluid is used in the damper, as the gas pressure can be tailored to urge movement in the desired axial direction with a desired force, while the speed (and hence the amplitude) of anyh inadvertent movement in either direction is limited. Once again, a variety of variations and modifications may be provided. While schematically shown outside the cylinder portions, restricted flow path 552 will optionally extend through fixed separator 554, and a variety of orifice structures or other flow-path restrictions may be employed, including simply sizing the orifice through the fixed separator (through which the shaft passes) appropriately relative to the shaft diameter. While shown axially offset, the damper and drive cylinder portions may alternatively be concentric, with a hydraulic damper cylinder cross-section optionally being smaller than a pneumatic cylinder cross-section (as hydraulics may accommodate higher pressures than pneumatics). Still further combinations of the systems described herein may be employed, including using one or more single-channel pneumatic systems 520 to pneumatically actuate one or more associated spool valve(s) of hydraulic two-channel system(s), with a plurality of gas fluid channels from the driver optionally being used to control hydraulic actuation of an associated plurality of axial articulation members. Hence, while the hybrid fluidic/mechanical catheters and systems illustrated herein will often include the two-channel damped system of FIG. 30C, alternative hybrid devices and systems as described may also be provided.

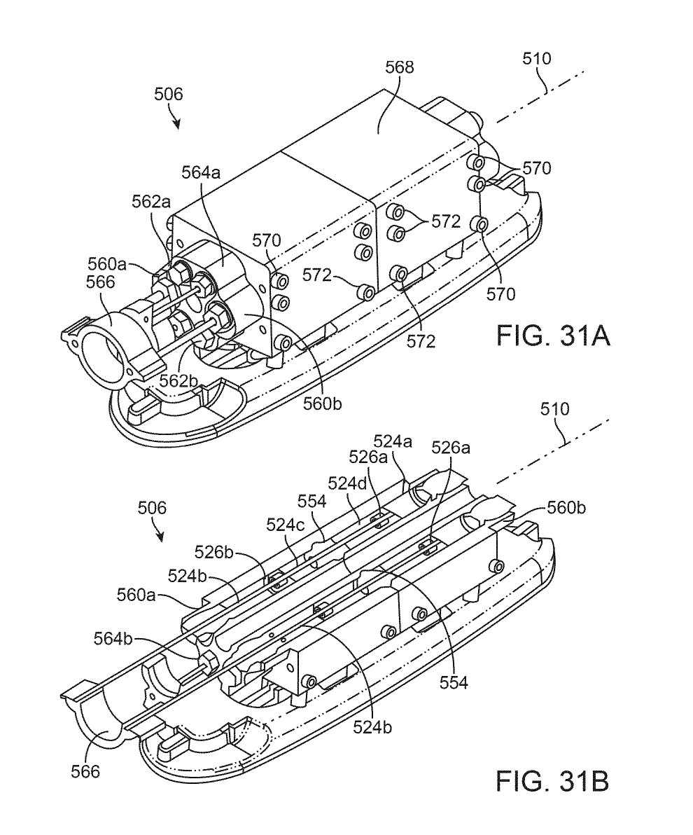

Referring now to FIGS. 29B, 31A and 31B, the exemplary piston system 516 of piston drive portion 506 in the proximal catheter housing includes 6 multi-piston cylinders 560a, 560b, 562a, 562b, 564a, and 564b arranged in 3 pairs, each pair being symmetrical about catheter axis 510. The axial load capacity of the individual cylinder/piston assemblies with a pair are combined and can be applied asymmetrically to the catheter shaft assembly, as the output shafts within each pair of pitons is affixed together by a yoke 566. Fill and drain fluid can be coupled through the wall of cylinder housing 568 by tubes (not shown) affixed to connectors 570, and the damper fluid may be introduced through damper access screws 572, as shown in FIG. 31A. The fill/drain channels for a cylinder portion are combined into a single tube, and the corresponding channels for the corresponding cylinder portions for cylinders within a pair are in fluid communication (e.g., using a common drain valve and a common fill valve) as the shafts for that pair will be driven together in parallel. The pistons 526a, 526b and fixed separators 554 of the piston system for one pair of cylinders 560a, 560b can be seen in FIG. 31B. The cross-sectional sizes of the cylinders may be consistent or may vary (as shown) so as to accommodate differing axial articulation loads of the catheter system. The load capacity for even a single cylinder can be quite high, typically being over 2 lb, often being over 5 lb, in many cases being over 10 lbs, and optionally being over 20 lbs. The load capacity for the paired cylinders will often be twice that of a single cylinder, so that with fluid pressures of up to or over 20 Atm. forces well over 40, or even well over 60 lb more may be generated. Cylinder housing 568 may comprise a relatively easily machined or even printed polymer or metal; the pistons, shafts, and fixed separators may see significant loads, and may comprise high-strength polymers or metals. Seals for the pistons an fixed separators may be commercially available from a number of suppliers, including Bal Seal Engineering, Inc. of Colorado.

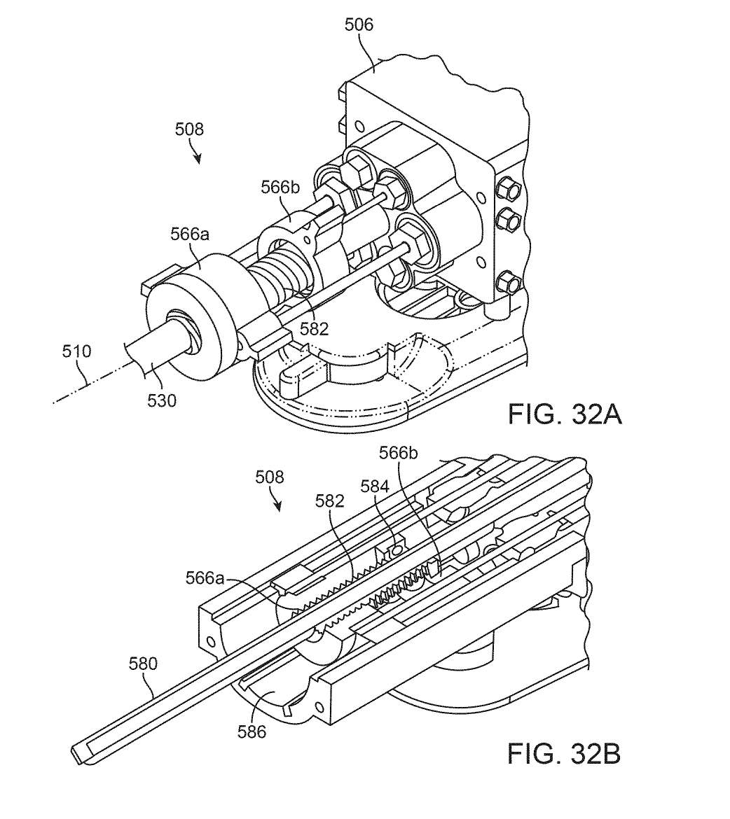

Referring now to FIGS. 32A and 32B, the structure and functionality of the rotation portion 508 of the proximal housing of the catheter can be understood. Rotation portion can induce axial motion or rotational motion or independently selectable combinations of both to a tubular catheter shaft of the catheter assembly. Rotation portion 508 extends distally of piston drive portion 506, and uses differential axial motion between two pairs of cylinders to rotate a shaft 580 of the catheter assembly about the catheter axis 510. More specifically, a first pair of output shafts from an associated pair of cylinders is affixed to a first yoke 566a. A second yoke 566b is similarly driven by another pair of cylinders of the piston drive portion 506. Second yoke 566b is axially affixed to a threaded body 582 by a bearing 584, so that the threaded shaft is free to rotate about catheter axis 510 relative to the second yoke. First yoke 566a has a threaded inner surface which engages the threads of threaded body 582, which is affixed to shaft 580. Hence, when first and second yokes 566a, 566b move axially together by the same distance and at the same speed and time, shaft 580 is driven axially with the two yokes at a fixed rotational orientation about axis 510. When second yoke 566b moves axially independently of yoke 566a, bearing 584 maintains axial alignment between shaft 580 and the second yoke 566b, so that the shaft moves axially with the second yoke. However, the rotational orientation of shaft 580 is determined by the engagement the threaded surfaces of the first yoke 566a and the threaded body 582. When the first yoke 566a moves axially relative to the second yoke 566b and shaft 580, the shaft still remains axially affixed to the second yoke, and the shaft is driven rotationally about axis 510. An axial slide housing 586 of the rotation portion 508 includes axially elongate positioning features that slidingly engage tabs of the yokes so as to accommodate axial motion of the yokes and maintain rotational and lateral alignment of the yokes relative to the catheter axis.

Referring now to FIGS. 29A, 29B, 32B, and 33A-33C, components and functionality of electromechanical portion 512 of the catheter housing can be more fully understood. Electromechanical portion 512 generally includes a plurality of motors 590 coupled to a plurality of pullwires 592a, 592b, and 592c by a gear and pully system 594. Motors 590 and the gear and pully system 594 are supported by a carrier 596, which is, in turn, supported axially by a third yoke 566c extending proximally from the piston drive portion 506 of the proximal catheter housing, allowing a pair of cylinder/piston assemblies to axially position and move these electromechanical drive components. Carrier 596 is axially coupled to third yoke 566c by a rotational bearing that allows the carrier to rotate relative to the yoke about the catheter axis. A non-axisymmetric shaft 580 extends proximally of the piston drive portion 506 and has a non-axisymmetric cross-section that is engaged by an inner surface of shaft 580 and by carrier 596 so that a rotational orientation of the carrier (and the components supported thereon) is driven by the rotation driver portion 508 via the shafts. Non-axisymmetric shaft 581 slides axially within shaft 580 so as to accommodate independent axial positioning of the shafts by the yokes.

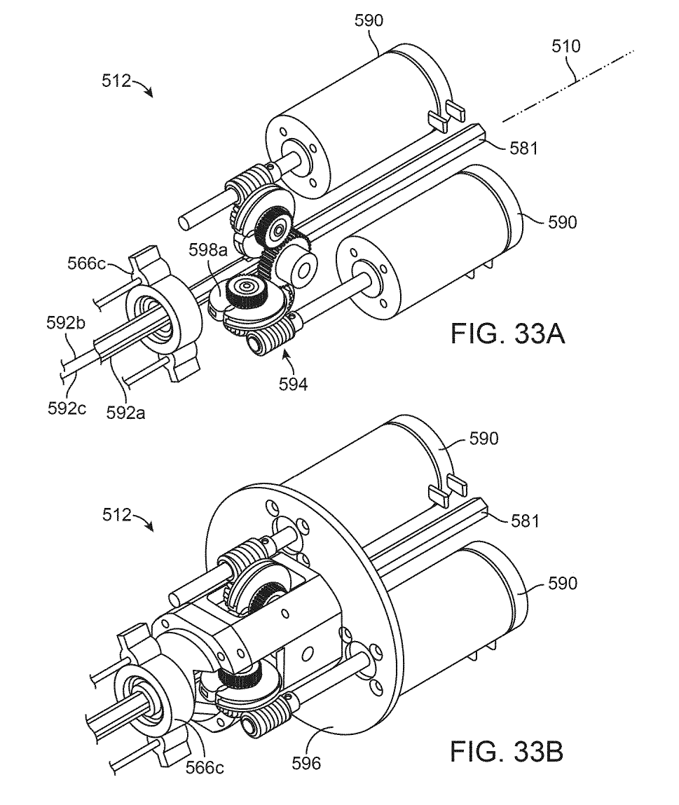

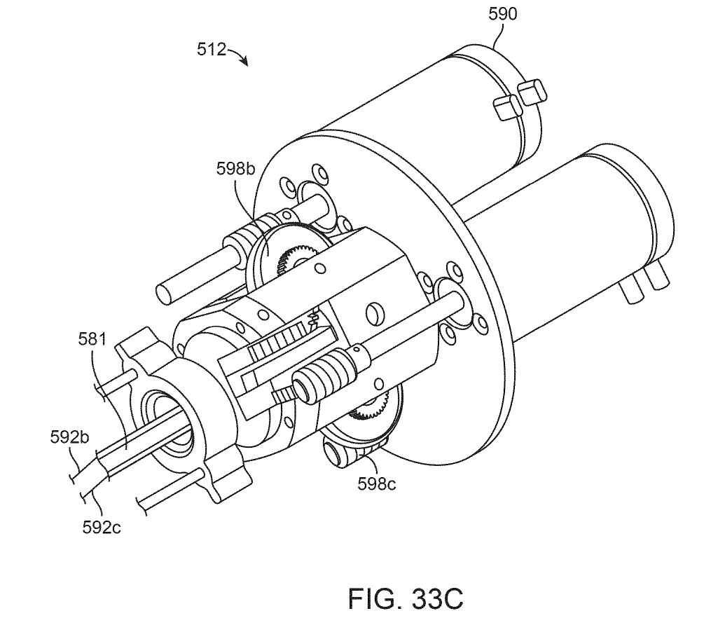

Referring to FIGS. 32B, 33A, and 33B, one of the motors drives a pully 598a via a worm gear so as to move a first pullwire 592a proximally, and to ease tension to allow it to advance distally. First pullwire 592a extends distally along an outer surface of non-axisymmetric shaft 581, and can be used to, for example, laterally deflect a distal portion of a catheter assembly (such as a distal end of shaft 580 or non-axisymmetric shaft 581) in the direction of the pullwire (relative to catheter axis 510) using a standard pullwire articulatable catheter structure along the distal portion. Note that the non-axisymmetric shaft may have a round profile distally of the proximal end of shaft 580. As the motor and other drive component are supported by yoke 566c and may be axially affixed to non-axisymmetric shaft 581, this allows pullwire 592a and its drive components to ride with the shaft as it moves axially and is rotated about the catheter axis. Use of pullwire 592a to articulate shaft 580 may benefit from active driving of pully 598a in response to (and so as to compensate for) relative movement between shaft 580 and non-axisymmetric shaft 581.

Referring now to FIGS. 33A and 33C, the other motor 590 of electromechanical portion 512 of the catheter housing drives opposed pullwires 592b, 592c via pullies 598b, 598c, respectively. The motor is again coupled to the pullies via a worm gear, and the two pullies 598b, 598c are coupled together by gears so as to rotate in opposite directions. Pullwires 592b, 592c extend distally within non axisymmetric shaft 581, and can be used to laterally deflect the distal portion of that shaft in opposed (e.g., +/−Y) lateral directions using well-known distal articulatable catheter shaft and pullwire structures. The opposed lateral articulation segment driven by opposed pullwires 592b, 592c will often be axially and circumferentially offset from the unidirectional lateral deflection segment articulated by guidewire 592a, analogous to the arrangement that can be seen (for example) in the manual articulated MitraClip delivery system.

As can be understood from the description above and the associated drawings, the hybrid systems described herein can use fluidic actuation of mechanical drive members, often via one or more pistons, to articulate a wide range of individual or nested flexible catheters or other flexible structures. The piston-driven articulatable features of these systems can make use of robotically controlled movements of pullwires, tubular shafts, or other laterally flexible mechanical articulation members with quite high force capabilities, and the stroke or axial movement of the mechanical members can be quite long (depending on the lengths of drive pistons or the like), with strokes often being between ½″ and 9″, more typically being from about 1″ to about 6″. These strokes can be used to articulate shafts, deploy prosthetic valves and other radially expandable structures (by withdrawing a sheath proximally while axially restraining the structure with a shaft disposed within the sheath), telescope an inner at least semi-rigid distal segment axially from within an outer at least semi-rigid segment, or the like. Such piston-driven articulation may also be combined with balloon array articulation, for example, using a piston-drive system to articulate, rotate, and axially position a relatively stiff guide catheter extending into or through the right atrium, with a balloon array articulated delivery system extending through the guide catheter being fluid driven within the left atrium and/or ventricle to position and orient a valve repair or replacement therapeutic tool for use. Some or all of these powered articulations may be robotically coordinated, and when desired, the user may manually manipulate components or tools through the delivery system so as to benefit from tactile feedback when interacting with tissues and the like. The components of the exemplary hybrid and balloon-articulated systems described herein can be selectively combined, for example, foregoing an electromechanical portion, replacing electromechanical articulation and rotation with a balloon array, or re-arranging the axial and rotational drive elements as appropriate for a particular therapy.

While the exemplary embodiment have been described in some detail for clarity of understanding and by way of example, a variety of modifications, changes, and adaptations of the structures and methods described herein will be obvious to those of skill in the art. For example, while articulated structures may optionally have tension members in the form of pull-wires as described above, alternative tension members in the form of axially slidable tubes in a coaxial arrangement may also be employed. Hence, the scope of the present invention is limited solely by the claims attached hereto.

What is claimed is:

1. A hybrid mechanical/fluid catheter system for treating a patient, the system comprising:
a flexible catheter assembly having a proximal catheter interface and a distal portion with an axis therebetween, an actuatable feature along the distal portion and a mechanical drive member extending proximally along the axis; and
a driver assembly having a fluid supply and a driver interface releasably coupleable with the catheter interface, the fluid supply operatively coupled with the driver interface such that drive fluid can articulate the catheter assembly when the catheter interface is coupled with the driver interface.

2. The catheter system of claim 1, wherein the fluid supply comprises a receptacle for a sealed cannister containing a liquid/gas mixture, wherein the catheter assembly comprises a catheter body having a distal catheter portion with an articulation balloon array and a plurality of lumens, each lumen being in fluid communication with an associated subset of the balloons.

3. The catheter system of claim 1, wherein the drive member comprises a pullwire or tubular shaft.

4. The catheter system of claim 1, wherein the catheter interface is disposed on a proximal housing supporting a first fluid-driven actuator, the fluid supply being coupled with the first fluid-driven actuator so as to actuate the actuatable feature in response to pressure from the fluid supply.

5. The catheter system of claim 4, wherein the driver interface has a first fluid channel and a second fluid channel, wherein the catheter interface has a first fluid channel and a second fluid channel configured for coupling with the first and second channels of the driver interface, respectively, so as to controllably drive the drive member in first and second opposed axial directions.

6. The catheter system of claim 4, wherein gas pressure is transmitted between the driver interface and the catheter interface, and wherein a damper is axially coupled with the first fluid-driven actuator, the damper containing a liquid and configured to damp axial movement of the drive member.

7. The catheter system of claim 4, wherein the first fluid-driven actuator comprises a first cylinder portion with a first piston axially movable therein, the fluid supply being coupled with the first cylinder, the drive member coupled with the piston, wherein the proximal housing contains a plurality of pistons movably disposed in a plurality of cylinders, a pair of the cylinders being axially coupled and laterally offset with the axis extending therebetween.

8. The catheter system of claim 4, wherein movement of the first fluid-driven actuator induces rotational actuation of the actuatable feature about the axis.

9. The catheter system of claim 1, further comprising a manual input configured to be moved by a hand of a user relative to the catheter interface so as induce movement of the driver.

10. The catheter system of claim 1, further comprising a sensor coupled with the drive member so as to provide feedback to a processor of the drive assembly, and/or one or more sensors coupled to the articulatable feature of the catheter.

11. A hybrid mechanical/fluid catheter for use in a robotic catheter system to treat a patient, the robotic system including a driver assembly having a fluid supply and a driver interface, the hybrid catheter comprising:
an elongate flexible catheter body having a proximal catheter interface and a distal portion with an axis therebetween, an actuatable feature along the distal portion and a mechanical drive member extending proximally along the flexible body, wherein the fluid supply is drivingly coupled with the actuatable feature by the mechanical drive member when the catheter interface is coupled with the driver interface.

12. A guide system for accessing and treating a mitral valve of a patient, the system comprising:
an elongate catheter body having a proximal end and an articulated distal portion with an axis therebetween, wherein a lumen extends along the axis;
a mitral valve treatment tool supported by the catheter body distally of the articulated portion;
a stiff guidewire receivable in the lumen of the catheter body so that the tool and articulated portion are advanceable over the guidewire, the guidewire having a proximal guidewire portion and a distal guidewire portion and configured to define a bend therebetween so that the distal portion extends primarily laterally relative to the proximal portion, wherein the proximal guidewire portion and the bend are sufficiently stiff that when the catheter body is advanced distally over the guidewire from adjacent the proximal end the guidewire bends the articulable portion primarily laterally relative to the proximal guidewire portion.

13. The system of claim 12, wherein the proximal guidewire portion and bend have a bending flexural stiffness of more than 50 GPa when measured using a 3-point bending test, and wherein the catheter body has an articulated distal portion, and further comprising a fluid driver couplable to the articulated distal portion so as to induce articulation.

14. The system of claim 12, wherein the guidewire comprises a pre-bent guidewire having the bend when at rest, and wherein the catheter body has a stiff catheter body portion proximal of the articulable portion, the stiff catheter body portion having a laterally stiffness greater than that of the guidewire along the bend so that the catheter body, when the bend is pulled proximally into the lumen along the stiff catheter body portion, reduces an angle of the bend to less than ½ a resting angle of the bend, wherein the pre-bent guidewire has an autramatic soft distal portion distal of the bend, and wherein the catheter body has a profile of more than 17 Fr.

15. A telescoping transseptal access system comprising:
an elongate catheter body having a proximal end and distal end with an axis therebetween, wherein a lumen extends along the axis, an at least semi-rigid catheter segment disposed near the distal end, and an articulatable body portion is proximal of the rigid segment, the rigid segment having a rigid segment length;
an extension catheter having an at least semi-rigid extension with an extension length corresponding to the length of the rigid segment of the catheter body, and a laterally flexible body portion proximal of the rigid extension so that the flexible body can move axially through a bend of the articulable portion, the extension fittingly slidable in the rigid segment such that the rigid extension can telescope distally therefrom.

16. The telescoping system of claim 15, wherein the extension catheter has an extension lumen, and further comprising a needle body slidably disposed in the extension lumen, the needle body comprising a tissue penetrating distal tip, an at least semi-rigid needle shaft slidably disposable in the rigid extension, and a flexible needle body portion proximal of the rigid needle so that distal advancement of the needle body from adjacent the proximal end can telescope the needle shaft from the extension to penetrate tissue with which the articulable segment aligns the rigid segment of the catheter body, wherein the extension has a dilation tip tapering radially inwardly distally so as to facilitate advancing of the extension over the needle through a wall of a heart, and further comprising a dilation balloon disposed on the extension proximally of the dilation tip, the dilation balloon having, in an inflated configuration, a proximal end configured to fittingly engage a distal end of the catheter body so as to have a sufficiently smooth outer transition to facilitate axial advancement of the catheter body into the balloon dilated wall of the heart, wherein the articulatable body portion has X and Y steering such that it is configured to be articulated in a first lateral bending orientation from outside the patient, and in a second lateral bending orientation from outside the patient, the second bending orientation being transverse to the first bending orientation, wherein the articulatable body portion comprises an articulation balloon array, and wherein the rigid segment length is between about 1.75 cm and about 4 cm.

17. A hybrid transseptal catheter system comprising:
a guide catheter body having a proximal end and a first articulatable portion with an axis therebetween, wherein a tension member extends from the first articulatable portion toward the proximal end so as to vary a bend of the first articulatable portion from outside a patient body when the guide catheter is in use; and
a positioning catheter body extendable distally from the articulatable portion of the guide catheter body, the positioning catheter body having a proximal portion supported by the guide catheter body and a distal end with a second articulatable portion therebetween, the second articulatable portion having an articulation balloon array.

18. The hybrid system of claim 17, wherein the guide body has a first stiffness and the positioning body has a second stiffness less than the first stiffness, the articulation balloon array providing the articulatable portion with X and Y steering such that it is configured to be articulated in a first lateral bending orientation from outside the patient, and in a second lateral bending orientation from outside the patient, the second bending orientation being transverse to the first bending orientation.
